(19) United States
(12) Patent Application Publication
(10) Pub. No.: US 2025/0001067 A1
(43) Pub. Date: Jan. 2, 2025

(54) Apparatus for large volume medication administration

(71) Applicant: SHL Medical AG, Zug (CH)

(72) Inventors: Martin Michael Coyne, III, Boonton, NJ (US); Christopher James Franzese, Boonton, NJ (US); Molly Christine Larson-Wakeman, Boonton, NJ (US)

(21) Appl. No.: 18/291,645

(22) PCT Filed: Jul. 28, 2022
(86) PCT No.: PCT/EP2022/071259

§ 371 Date: Jan. 24, 2024

Related U.S. Application Data
(60) Provisional application No. 63/226,494, filed on Jul. 28, 2021.
(60) Provisional application No. 63/226,498, filed on Jul. 28, 2021.
(60) Provisional application No. 63/226,499, filed on Jul. 28, 2021.

(51) Int. Cl. A61M 5/142; A61M 5/172; A61M 39/08

(52) U.S. Cl. CPC A61M 5/142; A61M 5/1723; A61M 39/08

(57) ABSTRACT

Apparatus, systems and methods are disclosed, which are configured to deliver a therapeutic medication to a patient. The apparatus, system and methods comprise a reservoir, a patient interface, a tubing set, and a fluid pump, and the components are configured to provide a calibrated flow rate based upon specific characteristics of the therapeutic medications passing through and internal lumen of the tubing set.

### CROSS REFERENCE TO RELATED APPLICATIONS

The present application is a U.S. National Phase Application pursuant to 35 U.S.C. § 371 of International Application No. PCT/EP2022/071259, filed Jul. 28, 2022, which claims priority to (i) U.S. Provisional Patent Application No. 63/226,494, filed on 28 Jul. 2021, (ii) U.S. Provisional Patent Application No. 63/226,498, filed on 28 Jul. 2021, and (iii) U.S. Provisional Patent Application No. 63/226,499, filed on 28 Jul. 2021. The entire disclosure contents of these applications are herewith incorporated by reference into the present application.

### TECHNICAL FIELD

Embodiments of the disclosure generally relate to apparatus and methods for large volume infusion of therapeutic medicines. Specific embodiments of the disclosure pertain to apparatus and methods configured to deliver a one or more therapeutic medications to a patient at a known, preselected, and controlled flow rate.

### BACKGROUND

Infusion and injection are commonplace medical procedures used to deliver a wide variety of therapeutic medicines of interest for a variety of diseases. As used herein, “infusion,” “injection,” and “administration” are used interchangeably, taking place by subcutaneous (SC), intramuscular (IM), intravenous (IV), or enteral routes, also terms used interchangeably. Administration route is based on a specific medication's pharmacokinetic (PK) profile, formulation components, approved regulatory labeling, individual clinical judgment, or clinical necessity.

The SC or IM route is frequently used for administration of smaller volumes using prefilled syringes and autoinjectors. Biologic medicines are frequently administered via the SC route with these devices. However, medications with larger volumes are not suitable for these devices, and the IV route is typically chosen, generally in hospitals or outpatient clinics. Given the safety risks and patient burden of at-home IV administration, pharmaceutical companies and patients generally prefer at-home SC administration. SC administration is generally considered less invasive and more straightforward for patients. As physiologic uptake of medication is slower via the SC route, there is potential for improved tolerability compared to IV administration.

Given these significant advantages in safety, tolerability, and convenience, the pharmaceutical industry has invested heavily in transitioning formulations from IV to SC administration and medication administration from the clinic to the home setting. However, many large volume delivery devices such as syringe or volumetric pumps are intended for use only by trained healthcare professionals and are unsuitable for home use.

Ambulatory pumps for home use have been developed that provide an alternative to hospital-grade devices. However, they require configuration by a healthcare provider, aseptic assembly of components by patients, and may not work properly if specific components are unavailable or inadvertently substituted. These errors may lead to infection, medication errors, and serious adverse events. As a result, applicability of these devices is limited. To fully realize the benefits of large volume administration in the home setting, there is a need for simple, error-proof, safe, and intuitive delivery devices suitable for use by patients who are not trained healthcare providers.

While SC administration is highly preferred by pharmaceutical companies and patients, not all medicines are readily transitioned from IV-to-SC administration. Bioavailability is determined through in-human clinical trials, is molecule-specific, and generally lower for the SC route versus the IV route. For the same molecule, larger SC volumes are likely required to provide equivalent bioavailability compared to IV delivery. However, these volumes may exceed the capacity of current large-volume SC devices, such as on-body injectors (OBIs), which are supplied in fixed volume increments, such as 3 mL, 5 mL, 10 mL, 25 mL, and 50 mL. Should volume requirements exceed available OBI devices, or require customization of an OBI, follow-on clinical trials or commercial launch of medications using the OBI may be delayed.

Individual medications are often part of a larger regimen of medicines, with standardized regimens corresponding to a specific disease state, treatment regimen, or medication. In a clinic setting, order sets contain all the information required to administer a standardized regimen. For example, an oncology regimen might include pre-medications, oncology treatments, and post-medications, all contained in an order set. Existing drug delivery devices are designed to administer a single medication and cannot support delivery of multi-medication regimens, limiting the ability to move therapy from the clinic to the home setting. There are no delivery devices that can detect and respond to a suspected infusion reaction, making administration of certain medications currently infeasible in the home setting and confining these medications to in-clinic delivery.

Furthermore, medication order sets may direct clinical staff to perform specific patient monitoring and permit contingent administration of emergency medication. This is particularly important for medications that cause infusion-related reactions in certain patients. Infusion reactions are potentially fatal, systemic reactions related to mode of action of the medication. Systemic infusion reactions are clinically distinct from localized injection site reactions or erythema from administration of a single agent such as would occur with an autoinjector, prefilled syringe, or OBI device, which are uncomfortable but not life-threatening. They demand an immediate halt to medication administration and administration of one or more counteracting medications. However, prior art devices neither allow detection of systemic infusion reactions nor delivery of emergency medication and cannot be safely used to administer medications where systemic infusion reactions could occur. This is a particular concern for biologic therapies and is especially relevant to oncology treatments.

In the clinic setting, administration of a medication regimen, associated monitoring, and clinical decision-making are documented in the patient's record within an electronic health record (EHR) system. The purpose of the EHR is to provide a complete clinical record of care for a patient, and safely manage medication regimens without relying on human memory or introducing human error. Healthcare providers update and review the EHR system in real-time for a given patient. Current drug delivery devices for home use do not have EHR interfaces, preventing their use with multi-medication regimens, contingent medication administration, or specific patient monitoring requirements. Moreover, administration of medication via other drug delivery devices, such as OBIs, may not be reflected in an EHR system.

In the clinic setting, EHR systems also provide vital patient safety functions. EHR systems ensure patients may safely receive certain medications based on physical vital signs, laboratory testing values, or administration of prior medications as scheduled. However, prior art delivery devices used in the home setting are focused on a single medication, lack integration into EHR systems, and thus cannot provide safety interlocks that are present in the clinic. As a result, present devices cannot prevent administration of medications in unsafe conditions.

Accordingly, there is a need for allowing administration of other medications before, during, and after the therapeutic medication, even if outside the clinic setting. There is also a need for drug delivery systems which do not impose arbitrary volume restrictions or “breakpoints” upon the drug development process and decouple formulation development and clinical trials from delivery device, apparatus and system design. Furthermore, there is a need for drug delivery devices, apparatus and systems that are configured for detecting system infusion reactions through specific sensors, arresting delivery of a medicine, and administering one or more emergency counteracting medications. There is also a need for apparatus, systems and methods that provide EHR integration, advance the art of drug delivery devices, apparatus and systems by allowing home delivery of complex regimens as ordered, updating administration in a patient's record, and allowing healthcare providers to review a complete regimen history for a patient without extra effort. There is also a need to provide apparatus, systems and methods that allow integration with an EHR system and only allowing administration of medications under safe conditions, replicating the safety measures at home that are currently present in clinic settings.

### SUMMARY

One or more embodiments of the disclosure are directed to an apparatus configured to deliver one or more therapeutic medications to a patient, the apparatus comprising a one or more reservoirs containing therapeutic medications; a patient interface configured to deliver contents of the reservoir into the body of the patient; a flexible tubing set in fluid communication with the reservoirs at the proximal end, and the patient interface at the distal end; and a fluid pump configured to expel the therapeutic medication from the reservoirs through the flexible tubing set and into the patient interface, wherein the flexible tubing set comprises a predetermined length and one or more internal medication lumens comprising a consistent internal diameter, the flexible tubing set configured to establish a specific, calibrated flow rate based on specific characteristics of the therapeutic medications passing through the internal lumen, the specific characteristics selected from the group consisting of viscosity, shear thinning behaviors, shear thickening behaviors, desired delivery time to the patient, and combinations thereof. In some embodiments the apparatus is modular. In some embodiments, the apparatus is configured to deliver the therapeutic medication to the patient at a known, preselected, and controlled flow rate. In some embodiments, the apparatus is configured to deliver the therapeutic medication to the patient at a known, preselected maximum flow rate. In some embodiments, the apparatus is configured to deliver a first medication at a known, preselected, and controlled first flow rate through a first lumen, and to deliver a second medication at a known, preselected, and controlled second flow rate through a second lumen, wherein the first flow rate is faster than the second flow rate.

Additional embodiments of the disclosure are directed to an apparatus configured to deliver a therapeutic medication to a patient, the comprising one or more reservoirs, each of the one or more reservoirs containing a therapeutic medication; one or more reservoirs containing a pre-medication to be administered before or a post-medication to be administered after the one or more therapeutic medications; a patient interface configured to expel contents of the reservoirs into the body of the patient; a flexible tubing set in fluid communication with the reservoirs at a proximal end of the flexible tubing set, and a patient interface at a distal end of the flexible tubing set; and a fluid pump to expel the therapeutic medication from each of the one or more reservoirs through the flexible tubing set and into the patient interface, wherein the flexible tubing set is provided with predetermined length and internal lumen of consistent internal diameter to provide a specific, calibrated flow rate based on characteristics of the therapeutic medications passing therethrough, the characteristics selected from the group consisting of viscosity, shear thinning behaviors, shear thickening behaviors, desired delivery time to the patient, and combinations thereof.

Further embodiments are directed to an apparatus configured to deliver one or more therapeutic medications to a patient, the apparatus comprising one or more reservoirs containing one or more therapeutic medications; an emergency reservoir containing an emergency medication; a patient interface configured to expel contents of the one or more reservoirs and the emergency reservoir into the body of the patient; and a flexible tubing set in fluid communication with the one or more reservoirs at a proximal end of the flexible tubing set, and the patient interface at a distal end of the flexible tubing set, wherein the flexible tubing set is provided with predetermined length and internal lumen of consistent internal diameter configured to provide a specific, calibrated flow rate based on characteristics of the therapeutic medications passing therethrough, the characteristics selected from the group consisting of viscosity, shear thinning behaviors, shear thickening behaviors, desired delivery time to the patient and combinations thereof.

Further embodiments are directed to an apparatus configured to deliver one or more investigational medicines during a clinical trial at one or more controlled flow rates, the apparatus comprising one or more reservoirs, each of the one or more reservoirs containing an investigational therapeutic medication; a patient interface configured to deliver contents of the reservoirs into the body of the patient; a flexible tubing set in fluid communication with the one or more reservoirs at a proximal end of the flexible tubing set, and the patient interface at a distal end of the flexible tubing set; and a fluid pump configured expel the investigational therapeutic medication from the reservoir through the flexible tubing set and into the patient interface, wherein each of several the flexible tubing sets is provided with a predetermined length and an internal lumen of a consistent internal diameter to provide a specific, calibrated flow rate based on characteristics of the investigational therapeutic medications passing therethrough, the characteristics selected from the group consisting of dose, concentration, viscosity, shear thinning behaviors, shear thickening behaviors, desired delivery time to the patient and combinations thereof, the characteristics corresponding to one or more clinical trial study conditions.

Another aspect of the disclosure is directed to a method for delivering an investigational therapeutic medication to a patient at one or more controlled flow rates during a clinical trial of an investigational medicine, the method comprising providing a clinical trial kit comprising an investigational therapeutic medication, a reservoir, a fluid pump, and one or more flexible tubing sets, each of the one or more flexible tubing set corresponding to a specific controlled flow rate for a specific investigational therapeutic medication and associated with one or more clinical trial conditions; selecting a selected flexible tubing set from the one or more flexible tubing sets corresponding to an individual patient's clinical trial condition, as specified in a clinical trial protocol or randomization schedule; attaching a proximal end of the flexible tubing set to the fluid pump to establish fluid communication with the fluid pump; attaching a distal end of the flexible tubing set to a patient interface; and administering an investigational therapeutic medication to the patient.

In another embodiment of a method, a method of providing an optimized tubing set for delivery to a patient a therapeutic medication exhibiting substantially non-Newtonian characteristics delivered by a single pump unit at one or more known, preselected, and controlled flow rates is provided. The method comprises identifying one or more desired flow rates of the therapeutic medication for administration to a patient based on desired pharmacokinetics of the therapeutic medication; identifying one or more ambient temperatures at which delivery of the therapeutic medication will occur; conducting testing to identify a relationship between temperature, viscosity, and concentration of the therapeutic medication in a pharmaceutical formulation for delivery to the patient; specifying values of an internal diameter, a length, and interior surface roughness of an experimental tubing set associated with one or more of the desired flow rates, based on one or more of theoretical calculations and computational fluid dynamic analysis; characterizing a force required to propel the therapeutic medication exhibiting non-Newtonian characteristics through the experimental tubing set; experimentally determining a required fluid pump power to dispense the therapeutic medication within the experimental tubing set at a plurality of temperatures and flow rates; adjusting the values of the experimental tubing set to accommodate an observed flow rate versus a desired flow rate and selecting the optimized tubing set; and confirming the desired flow rate through the optimized tubing set.

### BRIEF DESCRIPTION OF THE DRAWINGS

FIG. 1A shows a simplified partial cutaway front view diagram showing anatomic location of patient interface components to effectuate intravenous medication delivery using four common vascular access devices (VADs) featuring terminating luer taper connections in accordance with one or more embodiments;

FIG. 1B shows a simplified partial cutaway front view diagram showing anatomic location of patient interface components to effectuate intravenous medication delivery using an implanted vascular access device (VADs) or “port” and Huber needles in accordance with one or more embodiments;

FIG. 1C shows a simplified partial cutaway front view diagram showing anatomic location of patient interface components to effectuate subcutaneous and intramuscular administration using a variety of straight in and angled needle placements in accordance with one or more embodiments;

FIG. 1D shows a simplified partial cutaway front view diagram showing anatomic location of patient interface components to effectuate placement of a soft, flexible administration cannula and provide subcutaneous and intramuscular administration in accordance with one or more embodiments;

FIG. 2A shows a block diagram of selected functional components implemented in the drug delivery apparatus to deliver three medications in accordance with one or more embodiments;

FIGS. 2B-1 through 2B-5 show block diagrams of selected functional components implemented in the drug delivery apparatus to deliver combination therapy of several medications, illustrating exemplary administration sequences and time-delays based on regimen requirements, in accordance with one or more embodiments;

FIG. 2C shows a block diagram of selected functional components implemented in the drug delivery apparatus to deliver a therapeutic medication preceded and/or succeeded by certain other medications as part of a full medication regimen in accordance with one or more embodiments;

FIG. 2D shows a block diagram of selected functional components implemented in the drug delivery apparatus to deliver a medication of interest as well as various flushing solutions in accordance with one or more embodiments;

FIG. 2E shows a block diagram of selected functional components implemented in the drug delivery apparatus to deliver a medication of interest and contingently administer an emergency medication to counteract a systemic infusion reaction in accordance with one or more embodiments.

FIG. 3A shows a flow diagram of a clinical trial study process, illustrating how the present disclosure is integrated therein in accordance with one or more embodiments.

FIG. 3B shows a flow diagram of a process within one embodiment to design and refine a tubing set to deliver a non-Newtonian therapeutic medication at one or more rates based on formulation characteristics, expected pharmacotherapeutic effect, and expected dosing regimens studied as part of a human clinical trial.

FIG. 3C shows a schematic diagram of the governing parameters to design and refine a tubing set to deliver a substantially non-Newtonian therapeutic medication given formulation characteristics in accordance with one or more embodiments.

FIG. 4 shows a block diagram of selected functional components implemented in the drug delivery apparatus to provide closed-loop monitoring of patient status in order to detect a systemic infusion reaction, and allow one or more appropriate clinical responses to the systemic infusion reaction in accordance with one or more embodiments.

FIG. 5 shows a flow diagram of a process of one embodiment for detecting and responding to a patient infusion reaction during or after administration of one or more therapeutic medications.

FIGS. 6A-C show a cross-sectional diagram of tubing sets and medication lumens in accordance with one or more embodiments.

FIGS. 7A-B illustrate a portion of a tubing set containing an inline filter and flow restrictor in accordance with one or more embodiments.

FIG. 8 shows a block diagram of selected functional components implemented in the drug delivery apparatus to provide clinical study data integrity for an investigational therapeutic medication in accordance with one or more embodiments.

FIG. 9 shows a block diagram of selected functional components to deliver one or more therapeutic medications to a patient.

FIG. 10A is a representative example of information in a medication order for a single medication contained within an electronic health record system.

FIG. 10B is a representative example of information in a medication order set contained within an electronic health record system for administration of a medication regimen, including a variety of medication administration and other care instructions for a patient.

FIG. 10C shows a block diagram of selected functional components implemented in the drug delivery apparatus to provide association and verification of a drug delivery apparatus against a medication order in accordance with one or more embodiments.

FIG. 11 shows a further embodiment of the embodiment depicted in FIG. 9.

FIG. 12 provides a schematic of an embodiment of the decision-making algorithm within the controller 403, sensor(s) 407, and patient data 408 referenced in FIG. 4.

### DETAILED DESCRIPTION

Before describing several exemplary embodiments of the disclosure, it is to be understood that the disclosure is not limited to the details of construction or process steps set forth in the following description. The disclosure is capable of other embodiments and of being practiced or being carried out in various ways.

As used herein, “infusion,” “injection,” and “administration” are used interchangeably, taking place by subcutaneous (SC), intramuscular (IM), intravenous (IV), or enteral routes, also terms used interchangeably. Administration route is based on a specific medication's pharmacokinetic (PK) profile, formulation components, approved regulatory labeling, individual clinical judgment, or clinical necessity.

Embodiments of the disclosure provide apparatus, system and methods for medication administration wherein the number of medications, administration order, volume, delivery time, and route of administration are independently selected. Embodiments of the apparatus, systems and methods provide a single architecture usable from initial human clinical trials in a research facility through commercial launch in a home setting after drug approval. One or more embodiments provide for use in the home setting, where the apparatus, systems and methods are intrinsically safe and intuitive for use by a patient or lay caregiver without healthcare training.

Accordingly, embodiments of the disclosure provide drug delivery apparatus, systems and methods allowing delivery of many different medications, including those historically limited to in-clinic settings, in the home in a variety of sequences, rates, and settings. As will be appreciated by one skilled in the art, there are numerous ways of carrying out the examples, improvements and arrangements of devices, apparatus and/or systems disclosed herein. Although reference will be made to the exemplary embodiments depicted in the drawings and the following descriptions, the embodiments disclosed herein are not meant to be exhaustive of the various alternative designs and embodiments that are encompassed by the present disclosure.

Embodiments of the present disclosure advance the art of drug delivery devices, apparatus or systems by allowing administration of other medications before, during, and after the therapeutic medication, even if outside the clinic setting. One or more embodiments of the disclosure do not impose arbitrary volume restrictions or “breakpoints” upon the drug development process and decouple formulation development and clinical trials from delivery device, apparatus or system design. In addition, embodiments advance drug delivery devices, apparatus or systems by allowing detection of system infusion reactions through specific sensors, arresting delivery of a medicine, and administering one or more emergency counteracting medications. One or more embodiments of the disclosure further provide apparatus, systems and methods that provide EHR integration, advance the art of drug delivery devices, apparatus or systems by allowing home delivery of complex regimens as ordered, updating administration in a patient's record, and allowing healthcare providers to review a complete regimen history for a patient without extra effort. One or more embodiments provide apparatus, systems and methods that allow integration with an EHR system and only allowing administration of medications under safe conditions, replicating the safety measures at home that are currently present in clinic settings.

Various embodiments of the disclosure are directed to improved systems or apparatus and methods configured for large volume infusion of therapeutic medicines. More particularly, embodiments provide systems, apparatus and methods comprising components configured to be combined to deliver one or more therapeutic medicines via one or more physiologic routes of administration in sufficiently large and varying volumes to achieve a desired therapeutic effect. In one or more embodiments, therapeutic medicines may also optionally include pre-, post- and emergency medication administration to effectuate a complete therapeutic regimen as ordered by a healthcare professional. In some embodiments, the components utilized are part of a kit, and may be referred to as a kit of components. The systems, apparatus and methods of one or more embodiments are used to determine the pharmacologic and physiologic effects of one or more therapeutic medicines when the characteristics are unknown, and may be then used to deliver the therapeutic medicine(s) at the desired parameters to achieve the therapeutic effect when administered in a variety of settings, such as in-clinic or at-home. In addition, the system, apparatus and methods of one or more embodiments improve usability, safety, and convenience based on the administration setting and end user of the drug delivery device, apparatus or system.

One of more embodiments of the disclosure provides new and/or improved apparatus, systems, and methods for administering large volumes of parenteral or enteral medicines to a patient. Intravenous, subcutaneous, intramuscular, and enteral administration of large volumes are provided by the disclosure herein. More specifically, One of more embodiments of the disclosure allows medications currently limited to the clinic setting to be administered at home by patients or lay caregivers, without the need for highly trained healthcare professionals or clinic visits. As a result, One of more embodiments of the disclosure is ideally suited for home administration of large volume biologics, such as monoclonal antibodies.

Embodiments described herein provide drug delivery apparatus, system or methods with a configurable plurality of medication reservoirs to administer a variety of medication regimens, including multi-drug regimens, as are common in oncology. Regimens may be administered over time in a sequential, parallel, time-delayed, or contingent manner. In one or more embodiments, the drug delivery system is provisioned with an interface to an electronic health record system and one or more medication orders or order sets, allowing administration of a multi-drug regimens and contingent medication administration based on laboratory values or physiologic monitoring. One of more embodiments of the disclosure provides home administration of more complex medication regimens that exceed the capability of existing prior art devices.

In one or more embodiments, tubing sets are provided with restricted flow rates corresponding to one or more clinical trial conditions or dosing regimens for an approved medication. One of more embodiments of the disclosure also provides both pre-approval clinical trials and commercialized medicines to be administered with the same device, apparatus or system, greatly reducing cost, time to market, and device, apparatus or system complexity.

In one or more embodiments, reservoirs may be individually designed for short- or long-term drug stability, based on the medication regimen being administered with the device, apparatus or system. Reservoirs may be filled at point of use in the home by a patient or caregiver, by a dispensing pharmacy, or by a pharmaceutical manufacturer. Optionally, the drug delivery system may be configured with intravenous flush solutions before and after administration in some embodiments.

In one or more embodiments, the drug delivery system is provisioned with a controller, algorithm, and sensors coupled to the controller to detect a patient's potentially life-threatening systemic infusion reaction. Further, embodiments of the drug delivery apparatus, system and method can administer a countervailing emergency medication in response to a systemic infusion reaction autonomously or at the direction of a remote clinician monitor, permitting home administration of medications that would otherwise be confined to in-clinic administration due to monitoring requirements and safety considerations. Moreover, in one or more embodiments, the drug delivery system is configured to deliver prophylactic medications before and after a medication with propensity for causing infusion reactions.

In one or more embodiments, the drug delivery system is provided with an input/output interface to a clinical trial data management system. In some embodiments, the data management system contains permanent storage for data collected during the clinical trial from one or more drug delivery systems herein. In some embodiments, data within the permanent data storage is used to support a regulatory submission for drug approval. In some embodiments, one or more drug delivery apparatus or systems is associated with one or more investigational therapeutic medications and/or clinical trial administration conditions for a specific patient.

### Patient Interface

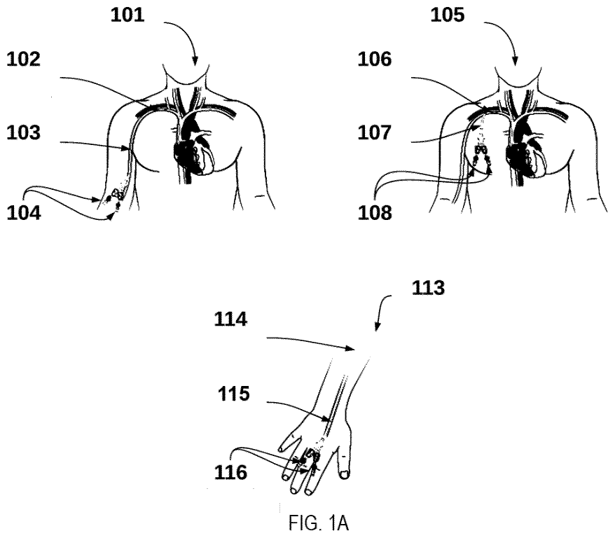

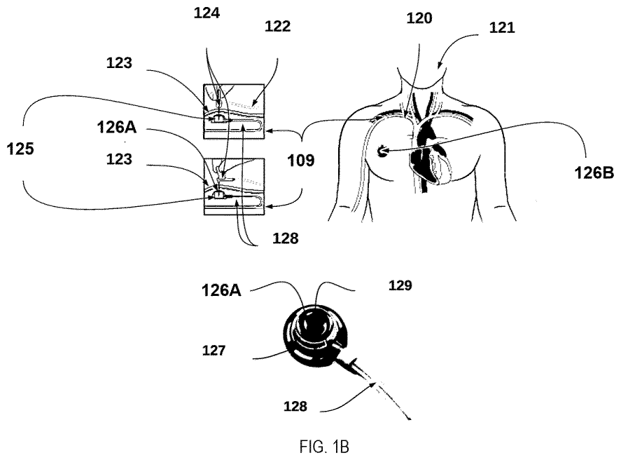

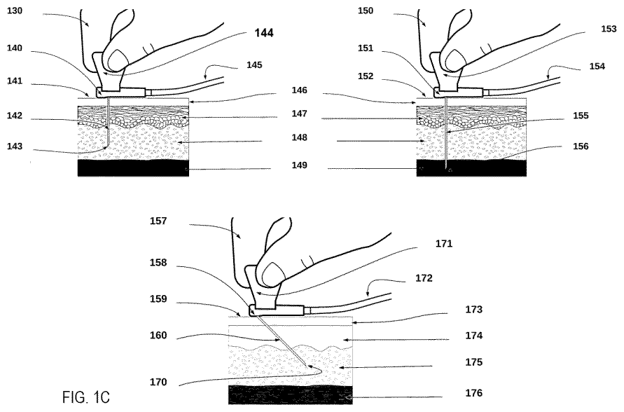

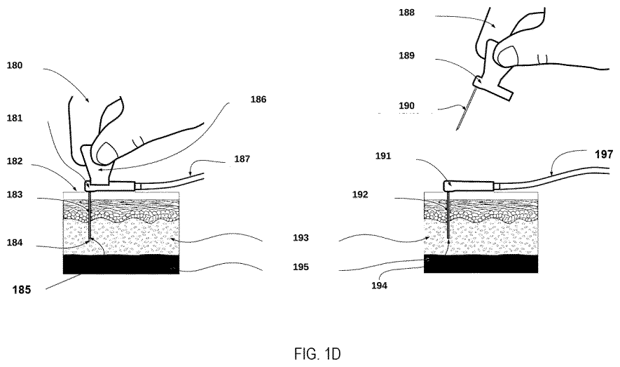

Selection of the physiologic administration route dictates the patient interface used to deliver medication to the patient. While the most common physiologic routes are shown in FIGS. 1A through 1D, many other configurations of a patient interface will be apparent those skilled in the art, and descriptions herein are for illustrative purposes only, and shall not be construed as limiting the present disclosure.

Referring to FIGS. 1A and 1B, for patients receiving medication via a peripheral intravenous catheter (PIV) 115 or central venous access device (CVAD) 107 and 103, the patient interface 104 is provided by means of a Luer-Lok® or luer taper connection familiar to those skilled in the art. For patients receiving medication via an implanted venous port 127 and catheter 128, the patient interface 125 is provided by means of percutaneous access to the needle entry septum 129 with a specialized steel needle, such as a Huber needle 124.

Referring to FIG. 1C, for patients receiving medication via the subcutaneous route the patient interface comprises a subcutaneous (SC) needle assembly 140 and 158 placing needles at 90° 142 or 45° 160 to the injection site, thereby accessing to the SC tissue 148 and 175, through hollow-bore needle points 143 and 170. For intramuscular (IM) administration with embodiments of the apparatus or system, the patient interface comprises an IM needle assembly 151, wherein a hollow-bore needle 155 is placed into the patient's muscle tissue 149 through open needle point 156. The material of needles 142, 155, and 160 are siliconized rigid medical grade stainless steel common in the art. Medications are delivered to the patient via integral tubing sets 145, 154, and 172.

Referring to FIG. 1D, for patients receiving medication via the SC or IM routes the patient interface may alternately comprise a flexible soft cannula placed by a removable, rigid inserter needle. A needle assembly 181 is inserted against the patient skin 182 by a patient or caregiver 180, optionally using one or more insertion affordances 186. Upon placement against the patient skin 182, a first portion of needle assembly 181 is removed by the user 188, retaining a portion 191 in the skin comprising the soft, flexible cannula 192 with open tip 194. The first removed portion of the needle assembly 189 comprises the steel inserter cannula 190 and the insertion and removal affordances 189. The retained portion of the needle assembly 191 includes a tubing set 195 for medication administration to the patient's SC tissue 193. IM administration is also provided simply by increasing the length of the flexible cannula 183 and inserter needle 184 to place the open end of the flexible cannula 194 into the patient's muscular tissue 195. The material of the inserter needle 190 is rigid siliconized medical grade stainless steel, and the material of the flexible administration cannula 183 may be any biocompatible polymer, such as PFTE.

### Drug Delivery System Components

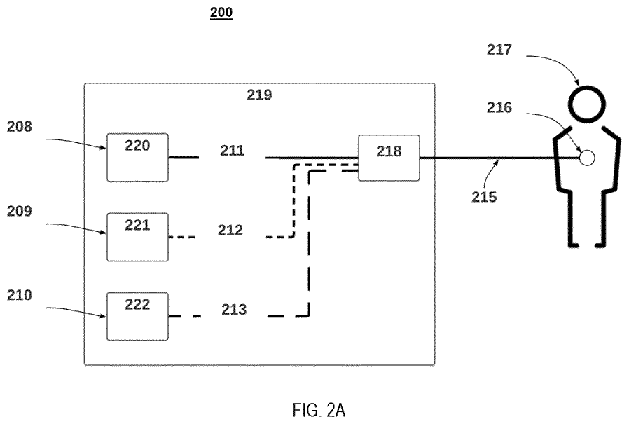

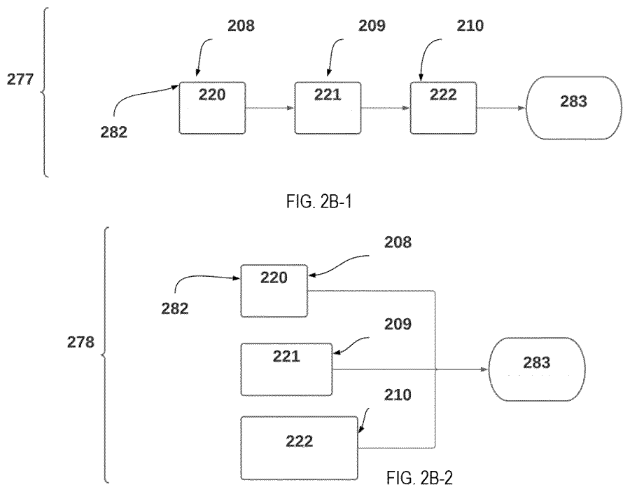

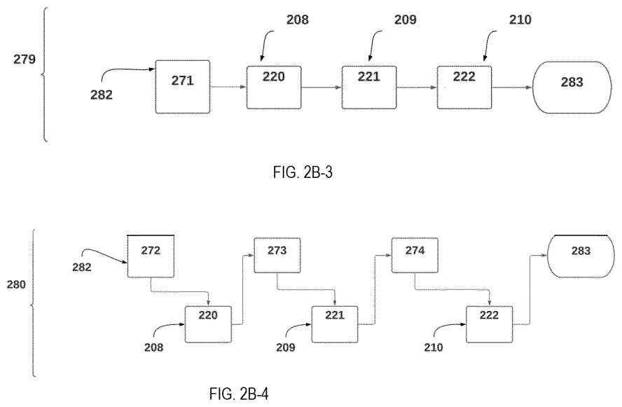

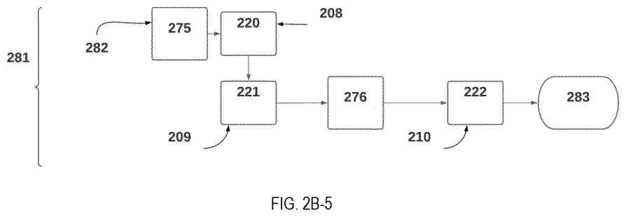

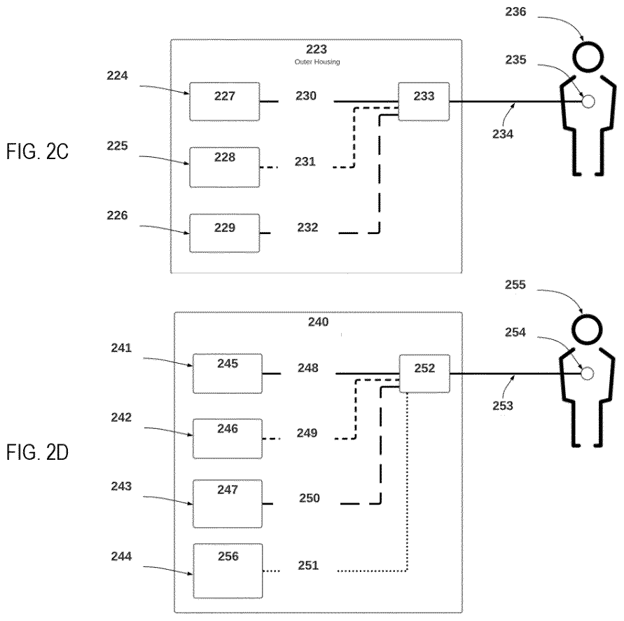

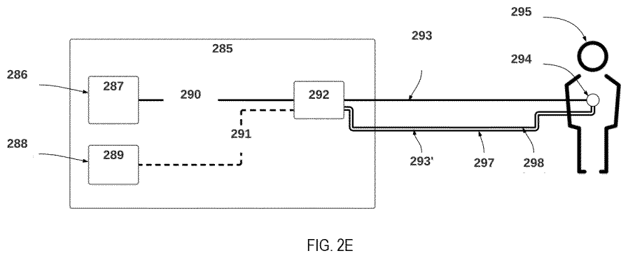

FIG. 2A illustrates variations of an exemplary drug delivery apparatus or system comprising an outer housing 219, a plurality of reservoirs 208, 209, and 210 for one or more therapeutic medication(s) 220, 221, and 222, fluidically connected 211, 212, and 213 to a fluid pump 218, by which the reservoirs may be emptied by the fluid pump 218 to administer the medication to the patient 217 by way of a tubing set 215 and patient interface 216. Although three reservoirs 208, 209, and 210 are described herein, many configurations of reservoirs are apparent based on the desired medication regimen, and are presented for illustrative purposes only, without limiting the present disclosure. As the exemplary embodiments make clear, any number of medications can be administered by the present system as desired.

In some embodiments, the outer housing 219 substantially encloses one or more reservoirs 208, 209, 210 and fluidic communication 211, 212, 213 between the reservoirs and fluid pump 218. In some embodiments, the outer housing 219 substantially encloses the fluid pump 218 and fluidic communication 211, 212, 213 between the reservoirs and fluid pump 218, and partially encloses one or more reservoirs 208, 209, 210.

In some embodiments, the outer housing is a rigid enclosure. In some embodiments, the outer housing is substantially flexible to conform to a patient's body or pocket. In some embodiments, the outer housing is configured with a single contoured side oriented towards and situated to conform to the patient's body. In some embodiments, the rigid plastic material, such as polypropylene, polycarbonate, acrylonitrile butadiene styrene, polyamide, or polystyrene. In some embodiments, the outer housing is over-molded on the side closest to the patient's body with a soft, compliant material, such as thermoplastic elastomer or thermoplastic polyurethane. In some embodiments, the outer housing is provided with a soft, compliant gel material on the side closest to the patient's body. In some embodiments, the outer housing is configured with a clip to allow attachment to a patient's clothing, pocket, or belt.

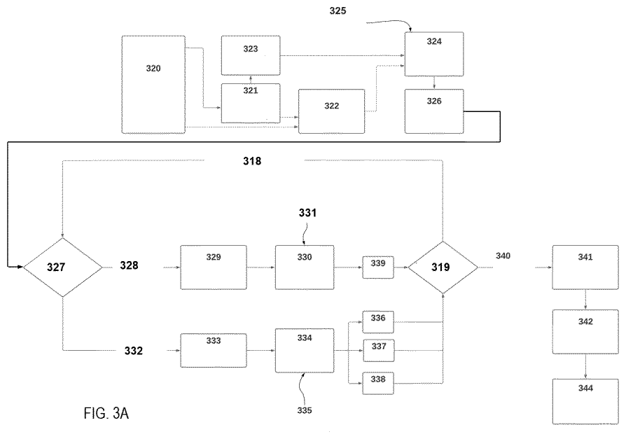

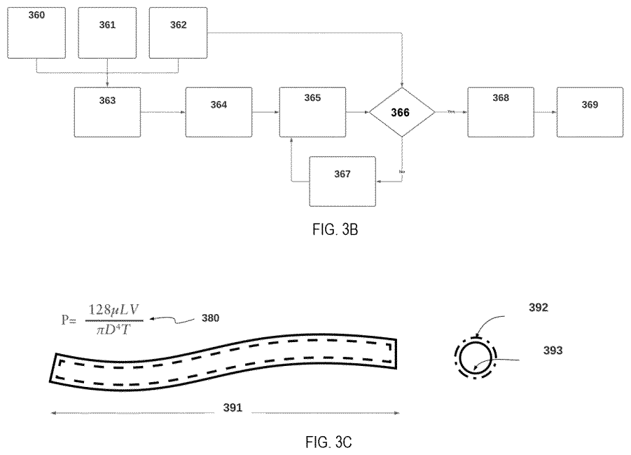

Referring to FIG. 2B, embodiments of the present drug delivery apparatus or system provide sequential, concurrent, time-delayed, and contingent administration of a variety of medications in a time sequence with a beginning 282 and end 283. During the time sequence, a plurality of medications 220, 221, 222 may be delivered in a prescribed sequential order 277 (as shown in FIG. 2B-1), in a concurrent manner 278 (as shown in FIG. 2B-2), in a prescribed sequential order 279 (as shown in FIG. 2B-3) in beginning after a prescribed time-delay 271, or in a in a prescribed sequence 280 (as shown in FIG. 2B-4) separated by one or more equally or unequally spaced time-delays 272, 273, and 274. Alternatively, during the time sequence, a plurality of medications 220, 221, 222 may be delivered in a prescribed sequence 281 (as shown in FIG. 2B-5), wherein certain medications are administered concurrently 220 and 221 after an optional time delay 275, after which other medications 222 are administered after a prescribed time-delay 276. The foregoing examples are for illustrative purposes and shall not be construed as limiting the number of medications or configurations that will be apparent to those skilled in the art.

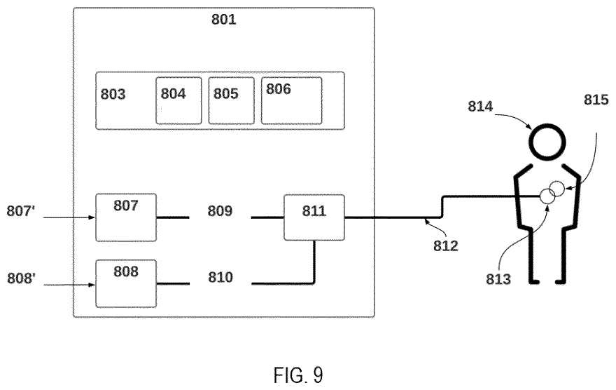

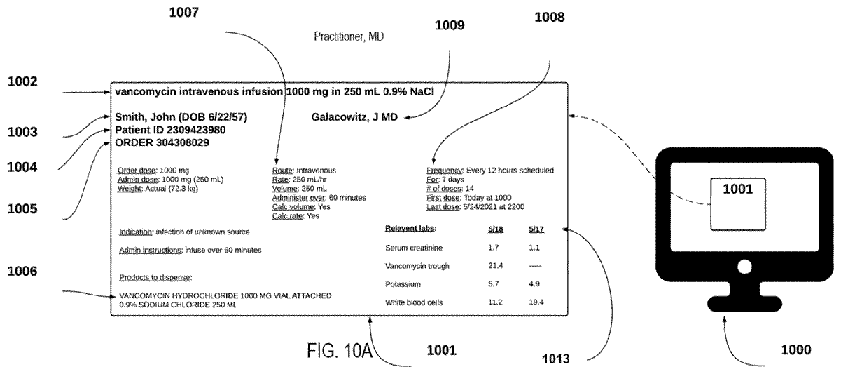

FIG. 9 illustrates variations of an exemplary drug delivery apparatus or system comprising an outer housing 801, a plurality of reservoirs 807′, 808′ for one or more therapeutic medication(s) 807, 808 fluidically connected 809, 810 to a fluid pump 811, by which the reservoirs 807′, 808′ may be emptied by the fluid pump 811 to administer the medication 807, 808 to the patient 814 by way of a tubing set 812 and patient interface 813. Although the plurality of reservoirs 807′, 808′ shows only two reservoirs, this is for illustration only, and the apparatus and systems described herein are not limited to a particular number of reservoirs. In one or more embodiments there can be any suitable number of reservoirs. The drug delivery system is also provided with a controller 803 that communicates with components of the apparatus via either a wired or wireless connection. In one or more embodiments, the controller according to one or more embodiments comprises a processor 804, a memory coupled to the processor 805, input/output devices 806 coupled to the processor 805 and support circuits to provide communication between the different components of the system, namely the components of the system described herein. In one or more embodiments, processes to operate the system are stored in the memory 805 as a software routine that, when executed by the processor, causes the system to perform methods described in the present disclosure. In one or more embodiments, the processes to operate the system are performed in hardware. In one or more embodiments, the software routine to operate the system may also be stored and/or executed by a second processor that is remotely located from the hardware being controlled by the processor. In some embodiments, the second processor comprises a cloud computing service or server. In some embodiments, the second processor comprises a remote patient monitoring system used by a healthcare provider. In some embodiments, the second processor comprises an electronic health record (EHR) system interface. In some embodiments, the second processor comprises a clinical trial data management system interface. In some embodiments, the second processor comprises a smartphone, smart tablet, smart television set, or voice activated assistant.

In one or more embodiments, one or more input/output devices 806 comprises a light source that may be illuminated upon receiving instructions or a signal from the controller 803. In one or more embodiments, the light source is coupled to an optical conductor in the tubing set 812. In one or more embodiments, one or more input/output devices 806 comprises a power source electrically coupled to a conductor within the tubing set 812.

In one or more embodiments, controller 803 may be also coupled to the fluid pump 811 to sense and/or control fluid flow therein. In one or more embodiments, controller 803 may be also coupled to one or more fluidic connections 809, 810 sense and/or control fluid flow therein. In one or more embodiments, controller 803 may be also coupled to one or more sensors and reservoirs 807′, 808′ containing medication 807, 808. In one or more embodiments, the outer housing 801, reservoirs 807′, 808′, and/or tubing set 812 may be configured with sensors also coupled to the controller 803. In one or more embodiments, controller 803 may be also coupled to one or more sensors 815 on the patient 814.

### Therapeutic & Other Medications

Various medications may be delivered by the present disclosure, including therapeutic medications, prophylactic pre-medications, prophylactic post-medications, emergency medications, and flushing solutions. Thus, “therapeutic medication” is used as a term of convenience herein to distinguish medications used to treat a disease (e.g., an oncology agent) from other ancillary medications delivered by the system while administering a therapeutic medication (e.g., a premedication or saline flush).

In some embodiments, a therapeutic medication is for treating one or more diseases selected from the group of cardiovascular, gastrointestinal, autoimmune, immunologic, hematologic, oncology, endocrinology, and respiratory disease. In some embodiments, a therapeutic medication is a coformulation of one or more medications for treating one or more of the aforementioned diseases. In some embodiments, multiple therapeutic medications are provided as part of a combination therapy.

In some embodiments, one or more therapeutic medications is a small molecule drug, therapeutic protein, cytokine, hormone, blood product, biologic, monoclonal antibody, antibody-drug conjugate, bispecific antibody, fusion protein, chimeric antigen receptor T cell therapy, cell or gene therapy, oncolytic virus, or immunotherapy.

In some embodiments, one or more therapeutic medications is an immuno-oncology or bio-oncology medication. In some embodiments, one or more therapeutic medications is selected from the group of several proposed targets, such as immune checkpoints, cytokines, chemokines, clusters of differentiation, interleukins, integrins, growth factors, enzymes, signaling proteins, pro-apoptotic proteins, anti-apoptotic proteins, T-cell receptors, B-cell receptors, or costimulatory proteins.

In some embodiments, one or more therapeutic medications is selected from the group of proposed mechanisms of action, such as HER-2 receptor modulators, interleukin modulators, interferon modulators, CD38 modulators, CD22 modulators, CCR4 modulators, VEGF modulators, EGFR modulators, CD79b modulators, Trop-2 modulators, CD52 modulators, BCMA modulators, PDGFRA modulators, SLAMF7 modulators, PD-1/PD-L1 inhibitors/modulators, B-lymphocyte antigen CD19 inhibitors, B-lymphocyte antigen CD20 modulators, CD3 modulators, CTLA-4 inhibitors, TIM-3 modulators, VISTA modulators, INDO inhibitors, LAG3 (CD223) antagonists, CD276 antigen modulators, CD47 antagonists, CD30 modulators, CD73 modulators, CD66 modulators, CDw137 agonists, CD158 modulators, CD27 modulators, CD58 modulators, CD80 modulators, CD33 modulators, APRIL receptor modulators, HLA antigen modulators, EGFR modulators, B-lymphocyte cell adhesion molecule modulators, CDw123 modulators, Erbb2 tyrosine kinase receptor modulators, mesothelin modulators, HAVCR2 antagonists, NY-ESO-1 OX40 receptor agonist modulators, adenosine A2 receptors, ICOS modulators, CD40 modulators, TIL therapies, or TCR therapies.

In some embodiments, one or more therapeutic medications is selected from one of ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab.

In some embodiments, one or more therapeutic medications is a part of a multi-medication treatment regimen. In some embodiments, one or more therapeutic medications is a part of a multi-medication treatment regimen selected from the group of AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOPAD, 7+3, 5+2, 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VeIP, OFF, GTX, CAV, AD, MAID, AIM, VAC-IE, ADOC, or PE.

In some embodiments, one or more therapeutic medications is used for adjuvant chemotherapy. In some embodiments, the chemotherapeutic compound is used for neoadjuvant chemotherapy. In some embodiments, the chemotherapeutic compound is an alkylating agent, plant alkaloid, antitumor antibiotic, antimetabolite, or topoisomerase inhibitor, enzyme, retinoid, or corticosteroid. In some embodiments, the chemotherapeutic compound is selected from the group of 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

In some embodiments, one or more therapeutic medications is classified as a hazardous medication according to the Centers for Disease Control's “NIOSH List of Hazardous Drugs In Healthcare Settings” or as defined by US Pharmacopeia General Chapter <800> “Hazardous Drugs-Handling in Healthcare Settings.”

When administering certain therapeutic medications, prophylactic medicines may be administered to a patient before (pre-medication) or after a therapeutic medication (post-medication) to avoid systemic infusion reactions or ease discomfort from a therapeutic medication's side effects. The pre-medication and post-medications may also comprise part of a medication regimen or medication order set, described elsewhere herein.

FIG. 2C illustrates an exemplary drug delivery apparatus or system configured to administer certain prophylactic medicines in addition to one or more therapeutic medications also contained within the system. In one or more embodiments, the drug delivery apparatus or system 223 contains a plurality of reservoirs for medication 224, 225, and 226. In some embodiments, reservoir 224 contains one or more prophylactic pre-medications 227 administered before the therapeutic medication(s) 228. In some embodiments, administration of the therapeutic medication 228 can take place only after complete administration of required pre-medication 224. In some embodiments, reservoir 226 contains one or more prophylactic post-medications 227 administered after the therapeutic medication(s) 228.

In one or more embodiments, one or more reservoirs 224 or 226 contains one or more medications selected from the group of 0.9% normal saline, 0.45% normal saline, 5% dextrose in water, 5% dextrose in 0.45% normal saline, Lactated Ringer's solution, albumin, and crystalloid fluids containing added electrolytes, such as potassium.

In one or more embodiments, one or more reservoirs 224 or 226 contains one or more medications selected from the group of analgesics, antipyretics, corticosteroids, antihistamines, antiemetics, antibiotics, anticoagulants, fibrinolytics, or antithrombolytics. In one or more embodiments, one or more reservoirs 224 or 226 contains one of diphenhydramine, acetaminophen, ondansetron, or famotidine.

In one or more embodiments, one or more reservoirs 224 or 226 are configured to reconstitute a lyophilized pre-medication or post-medication contained in a dual-chamber syringe featuring a bypass chamber. In one or more embodiments, one or more reservoirs 224 or 226 are configured to reconstitute a lyophilized pre-medication or post-medication in an anticipatory fashion to allow more timely administration.

When administering medications intravenously, it is necessary to flush the IV catheter system before and after medication administration. Flushing refers to the process of instilling a fluid volume after therapeutic medication delivery through the entire IV system to ensure all medication within the IV system is fully administered to the patient and to prevent clotting of the catheter system. In one or more embodiments, the drug delivery apparatus or system may also be configured to deliver of a therapeutic medication in conjunction with catheter flushing protocols.

Referring to FIG. 2D, in one or more embodiments, the drug delivery apparatus or system is provided with flush reservoirs 241, 243, 244 and a reservoir for therapeutic medication 242. The delivery apparatus or system is configured to deliver one or more catheter flushing solutions before 245 and/or after 247, 256 administration of one or more therapeutic medications 246. In one or more embodiments, the delivery apparatus or system administers an 0.9% Normal Saline from a pre-administration flush reservoir 241 followed by one or more therapeutic medications 246 in a reservoir 242, followed by an 0.9% Normal Saline Flush in a first post-administration flush reservoir 243, followed by Heparin Lock Flush solution in a second post-administration flush reservoir 244. Flushing need not be limited to the beginning and end of an administration process; when multiple medications are administered, flush reservoirs may be interposed between therapeutic medication administrations if desired.

In some embodiments, one flushing solution is 0.9% Normal Saline. In some embodiments, one flushing solution is recombinant tissue plasminogen activator (r-TPA). In some embodiments, one flushing solution is one or more medications selected from the group of 0.9% Normal Saline, Heparin Lock Flush solution, 100 U/mL Heparin Lock Flush Solution, and 5000 U/mL Heparin Lock Flush Solution. In some embodiments, one flushing solution is an antimicrobial. In some embodiments, one flushing solution is an antimicrobial combined with an anticoagulant.

### Tubing Set

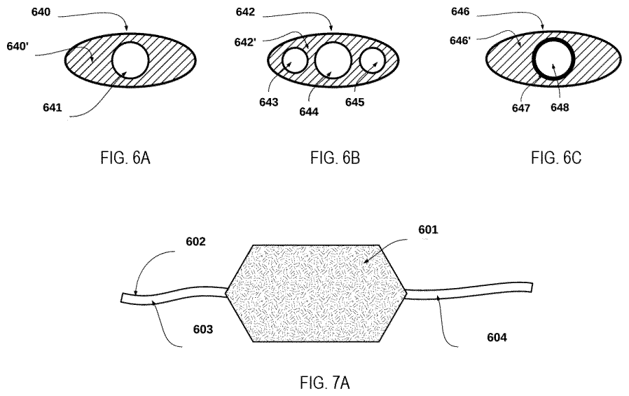

FIGS. 6A-C illustrate variations of an exemplary tubing sets for use with the present disclosure. In one embodiment, a tubing set 640 is provided with cross-sectional tubing profile 640′ and at least one inner medication lumen 641. During use of the drug delivery system, inner medication lumen 641 is in fluidic communication with a fluid pump and a patient interface described elsewhere herein to deliver medications within the system to the patient.

It may be desirable to isolate one or more inner medication lumens 648 from potential contaminant leachable or extractable compounds from the tubing set material, thereby improving compatibility with the medication delivered therein. Accordingly, in some embodiments, barrier coating 647 may be interposed between an inner medication lumen 648 and tubing set material 646′. In one embodiment, the barrier coating comprises a PTFE fluoropolymer material. In another embodiment, the barrier coating is co-extruded as the tubing set is manufactured. In another embodiment, the interior medication-contacting surface of one or medication lumens are provided with a hydrophobic coating.

It may be desirable to offer multiple flowrates in the present drug delivery system without switching tubing sets. Accordingly, in one embodiment, a tubing set 642 is provided with cross-sectional tubing profile 642′ and two or more medication lumens 643, 644, 645. The medication lumens may have different or similar diameters, thereby allowing administration of medications at flow rates in a variety of configurations. By way of example, the same medication administered through a first lumen 644 would flow more quickly than if administered through a second lumen 643 in the tubing set design exemplified in FIG. 6C. In an alternative embodiment, medication delivery may be accelerated by switching flow from a smaller to a larger lumen (e.g., from 643 to 645). In an alternative embodiment, medication delivery may be decelerated by switching flow from a larger to a smaller lumen (e.g., from 645 to 643). In an alternative embodiment, one or more medication lumens may be engaged in parallel fashion (e.g., using 643 and 645, or 644 and 643) to provide faster administration of a single medication. In an alternative embodiment, one or more medication lumens may each deliver a different medication concurrently. In an alternative embodiment, one or more medication lumens remains unused by the system until desired, as in the case of emergency medication administration as described herein.

Elements of the tubing sets described herein may take various shapes and forms. In one or more embodiments, cross-sectional tubing profiles may take a substantially circular, elliptical, rectangular, or polygonal shape. The flexible portion of the tubing set may be fashioned from one or more of silicone, PVC, PVC without DEHP, EVA, HDPE, LDPE, TPU, PTFE, a fluoropolymer, or other suitable flexible material. In one or more embodiments, tubing sets are extruded but may be formed by other means that provide sufficient dimensional and tolerance control on the inner medication lumens as described herein. In one or more an embodiments, the tubing material is chosen to be a material selected for low leachable and extractable compounds that may contaminate a medication, and that exhibits high biocompatibility with biologic medications.

Optionally, the flexible portion of the tubing set may comprise segments of one or more flexible materials, providing different degrees of flexibility at different sections along the length. For instance, a more rigid material may be provided near the connections to the fluid pump for strain relief and anti-kinking, while a more flexible material may be selected near the patient interface for comfort against a patient's skin. The exterior of the tubing set may be provided with a PFTE fluoropolymer or other permanently lubricious coating to prevent dragging or snagging of the tubing set on a patient's skin or clothing.

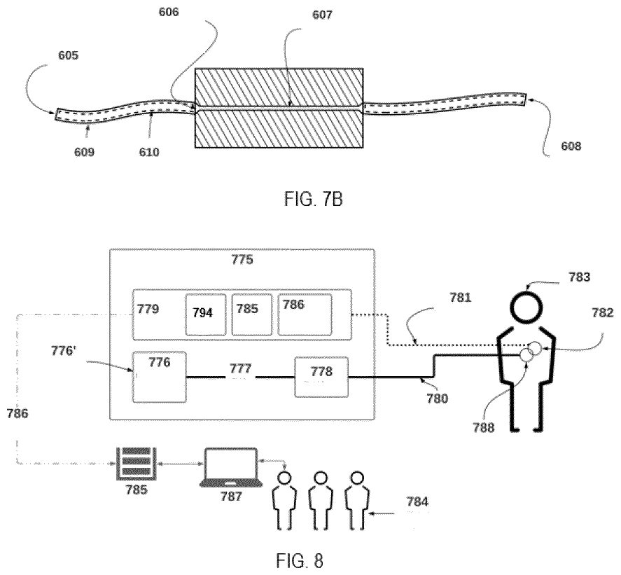

In one or more embodiments, and referring to FIGS. 7A-B, one or more tubing sets is provided with an inline filter 601 to remove undesirable or immunogenic particulate matter 602 prior from the inflow medication 603 prior to patient administration at the outflow side of the filter 604. The inline filter material is ideally selected to be low-sorbing, low protein binding, and compatible with the medication(s) therein. Optionally, the inline filter may comprise a multi-layer filter membrane, with each membrane layer featuring a different filter pore size.

In one or more embodiments, one or more tubing sets are provided with an engineered flow restriction 607 to provide an inflow medication 605 at a first rate, and outflow medication 608 at a second rate substantially less than the first rate. When used with biologic or shear-sensitive medications, the smoothed inlet 606 and engineered flow restriction 607 is in one or more embodiments designed to prevent protein damage or shearing.

### Fluid Pump

A variety of fluid pumps may be used in the disclosure herein, based on the configuration of reservoirs, viscosity of medications, and number of medications. In some embodiments, a single fluid pump is provided. In some embodiments, multiple fluid pumps are provided. In some embodiments, the fluid pump is configured to start, pause, or stop on demand. In some embodiments, the fluidic pump is configured with a transmission mechanism to provide selective engagement and disengagement of selected medication reservoirs. In some embodiments, the mechanical drive is coupled to a gear mechanism to reduce the form factor of the apparatus or system. In some embodiments, the gear mechanism comprises mating bevel gears. In some embodiments, the fluidic pump is prevented from operation if one or more medications is insufficiently viscous. In some embodiments, the fluidic pump is provided with a sensor to determine the temperature of a fluid at the fluid pump inlet.

Fluid pumps may be powered by, for example, a flat coil spring, wound helical spring, strip spring, pressurized gas, or an electrical motor. In some embodiments, a rotary power source may be coupled to one or more reservoirs through a worm screw and worm gear. In some embodiments, the worm screw and worm gear is used to hold a reservoir in a given position while other reservoirs are driven by the system. In another alternative embodiment, the fluid pump be driven by a power unit with rate control assembly, such as disclosed in U.S. Pat. No. 10,252,005. In another alternative, the fluid pump may be driven by a chemical engine, such as disclosed in U.S. Pat. No. 9,795,740. In another embodiment, the fluid pump may be drive by a power unit with progressive engagement mechanism, such as disclosed in U.S. Pat. No. 10,357,612. In another embodiment, the fluid pump may be driven by a rotary drive, such as disclosed in U.S. Pat. Nos. 8,617,109, 8,876,766, 9,022,982, 9,095,657, 9,132, 236, 9,446,201, 9,468,722, 9,737,668, 10,255,827, 10,307,543, 10,456,521, 10,507,289, 10,525,213, 10,632,248, 10,874,804, 10,881,811 and 11,065,387, the entire contents of each of these patent documents incorporated by reference in their entirety.

In an alternative embodiment, the fluid pump is a one-time use disposable design. In an alternative embodiment, the fluid pump is a reusable design for multiple medication administrations. In an alternative embodiment, the fluid pump is a reusable design designed to administer a single cycle of a medication regimen.

In one or more embodiments, one or more fluidic connections are designed to minimize internal volume that is not administered to the patient, thereby reducing medication waste and the need for medication overfill. Accordingly, in one or more embodiments, fluidic connections between one or more reservoirs and the fluid pump may comprise a manifold. In an alternative embodiment, each fluidic connection between one or more reservoirs and the fluid pump may have proportionally different relative to each other, permitting independent flow rate control of one or more medications beyond that provided by one or more tubing sets provided with the drug delivery system.

### Fluid Pump+Tubing Set Integration

In an embodiment, the fluid pump is sufficiently well-powered to deliver a full range of volumes, viscosities, and rates independently of the inner diameters of a tubing set, thereby allowing the same fluid pump design to be used for a variety of medications. This has the advantage of mass-producing fluid pumps and gaining efficiencies of scale. This approach allows design of a drug delivery apparatus or system without knowing medication formulation characteristics a priori. This is particularly important in clinical trials, where medication formulation characteristics are still in development, and dosing regimens are not yet finalized.

It is apparent that tubing sets in the present disclosure are used to control administration parameters for a therapeutic medication and accommodate flow characteristics of specific drug formulations without the need for complex or precise mechanical or electromechanical pumps. This is particularly important for biologic drug products or extended-release formulations displaying non-Newtonian shear-thinning and shear-thickening behaviors where modeling techniques are of limited usefulness.

FIG. 3B depicts an embodiment of a process to design tubing sets for use in a clinical trial in accordance with the disclosure herein. Formulation characteristics 360, pharmacokinetic modeling parameters 361, and desired clinical trial conditions 362 are inputs to initial numeric modeling 363 using either Hagen-Pouiselle's equation 380 (FIG. 3C) or other modeling methods, such as computational fluid dynamics. Modeling 363 provides initial design and component selection 364, comprising minimally first estimated nominal tubing lengths 391, tubing nominal internal diameters 392, and corresponding tolerances 393 on the nominal internal diameters 392 (FIG. 3C).

Tubing may be manufactured based on initial design and component selection 364. However, for non-Newtonian fluids, initial numeric modeling 363 may be substantially different than predicted, and adjustments to tubing internal diameters 392, and corresponding tolerances 393 on the internal diameters 392 may be required. The adjustments may require time-consuming or costly changes to extrusion dies or other equipment, and multiple testing and adjustment cycles may be required.

Regardless, the flow rate provided by the initially selected components 364 are physically tested 365 with the drug formulation of interest and compared to the desired clinical trial conditions 324, 330, 334, and 342. Physical testing 365 may optionally include characterization of any damage to the drug product caused by the tubing set or flow rates, including protein damage or shearing effects which may render protein-based medications inactive or harmfully immunogenic to humans. Physical testing 365 may optionally be conducted at temperatures representative of the administration setting for the final medication in clinical practice, which is especially relevant for medications that exhibit a nonlinear viscosity-temperature-concentration relationship, such as biologics.

As many medications display non-Newtonian shear-thinning and shear-thickening behaviors, empirical results may also differ from theoretical calculations, in which case components are iteratively redesigned 367. Individual tubing sets corresponding to a specific flow rate for a specific medication are individually analyzed, refining either tubing lengths 391 or tubing diameters 392, or specifying precision tolerances 393 on the diameters 392. Once precisely designed, a plurality of tubing sets is manufactured 368 for use with the overall drug delivery system to execute a given clinical study design 369 as previously specified.

### Medication Reservoirs

Referring to FIG. 2A in one or more embodiments, medication reservoirs 208, 209, and 210 are designed for short-term duration contact with therapeutic medications 220, 221, and 222, minimizing the technical burden and risk associated with long-term stability or container closure testing. In an alternative embodiment, the reservoirs 208, 209, and 210 are each selectively designed for short- or long-term drug contact based on the nature of the medicine 220, 221, and 222 therein.

Referring to FIG. 2C, in one or more embodiments, medication reservoirs 224 and 226 are long-term stability primary containers that are prefilled with medications 227 and 228, and reservoir 225 with a therapeutic medication 228 is designed for short-term stability and filled just prior to administration.

In one or more embodiments, one or more reservoirs is a glass or plastic syringe or cartridge prefilled by the manufacturer. In one or more embodiments, the interior surface of one or more reservoirs contains controlled levels of a silicone lubricant. Optionally, the silicone lubricant may be crosslinked, as through radiation. In one or more embodiments, one or more reservoirs is a single syringe with a plurality of reservoirs, chambers, or compartments.

In an embodiment of the of the present disclosure, one or more reservoirs is a flexible nonelastic container. In one or more embodiments, the flexible nonelastic container is fully emptied through application of a compressive force. Optionally, the flexible nonelastic container may be contained in a rigid protective shell. In one or more embodiments, one or more reservoirs is a flexible elastomeric container. In one or more embodiments, one or more reservoirs is a flexible container with one or more segments, each containing a single medication.

In some embodiments, one or more reservoirs are manufactured from one or more materials selected from the group of borosilicate glass, cyclic olefin polymer, cyclic olefin copolymer, PVC, EVA, fluorinated ethylene propylene (FEP) resins or films, PTFE, a fluoropolymer, or other suitable material. In other embodiments, one or more reservoirs are manufactured from a low-sorbing material. In some embodiments, one or more interior reservoir surfaces in contact with medication has a hydrophilic coating or has been passivated to reduce protein sorbing or formation of protein aggregates.

In some embodiments, the reservoirs are filled by pharmacy before dispensing to a patient. In some embodiments, the reservoirs are filled by a patient or caregiver at home. In some embodiments, the reservoirs are prefilled and assembled into the drug delivery system prior to use by a patient. In one or more embodiments, one or more reservoirs is filled while contained in the drug delivery apparatus or system. In one or more embodiments, one or more reservoirs is filled outside the drug delivery apparatus or system, then installed into the drug delivery system as a secondary operation. In one or more embodiments, one or more reservoirs is filled by the patient, lay caregiver, or healthcare provider. In an alternative embodiment, one or more medication vials are provided with a vial transfer apparatus or system for filling a reservoir. In an alternative embodiment, the reservoir is pre-attached to a transfer apparatus or system to effectuate filling with a minimum of use steps and corresponding risk of aseptic breach. In an alternative embodiment, the reservoir is filled from a vial using pressure applied by a compressed gas. In an alternative embodiment, the reservoir is filled from a vial using pressure applied by an electromechanical pump assembly.

In one or more embodiments, the drug delivery system is equipped with one or more features to prevent unauthorized access to, or diversion of, one or more reservoirs containing a controlled substance after filling. The features may include a tamper-evident seal on the exterior of the drug delivery apparatus or system or internal sensors to detect unauthorized access to the drug delivery system and components within it, including medication reservoirs.

In some embodiments, one or more reservoirs is provided with a sensor to determine the temperature of a fluid therein. In some embodiments, the sensor is located on the exterior of the reservoir. In some embodiments, the sensor is a temperature probe making direct contact with the medication through the reservoir wall.

### Infusion Reaction Detection

Used herein as a term of convenience, infusion reactions include standard infusion reactions (SIRs), cytokine-release reactions, or IgE-mediated allergic reactions. As new categories of biologics with novel modes of action are developed and commercialized, additional types of patient infusion reactions may also become apparent beyond those listed herein. Thus, the foregoing infusion reactions cited herein are provided by way of example and shall not be construed as limiting the scope of disclosure of the disclosure herein.

Certain medications are associated with overall higher incidence of infusion reactions. For these medications, specific pre- and post-medications are administered to reduce incidence of infusion reactions or negative patient impacts should they occur. Administration of pre- and post-medications is provided by the present disclosure as illustrated in FIG. 2C and described elsewhere herein.

However, even when prophylaxis is administered, infusion reactions can occur. Infusion reactions are clinically distinct from injection site reactions, which cause localized discomfort and are neither emergent nor life threatening to the patient. Onset of infusion reactions is sudden, systemic, and life-threatening; treatment requires unexpected and immediate administration of counteracting emergency medications. Due to rapid onset, healthcare providers monitor patients routinely in the clinic setting and intervene immediately.

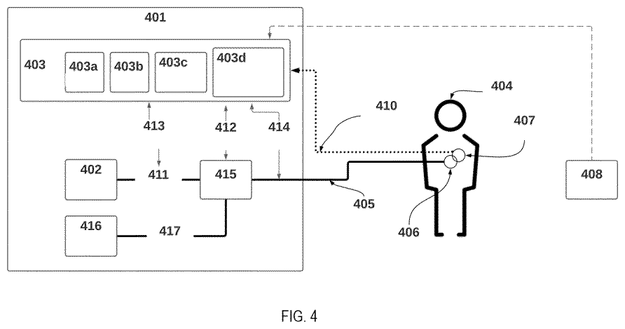

Due to the serious nature of infusion reactions, it is highly desirable to anticipate potential infusion reactions at onset, especially in settings outside the clinic, which is also provided by alternative embodiments of the drug delivery apparatus or system herein. FIG. 4 illustrates an exemplary drug delivery apparatus or system configured to include sensors to detect potential infusion reactions, a controller and algorithms, features to interrupt medication flow, and optional features for delivery of emergency medications in response to an infusion reaction.

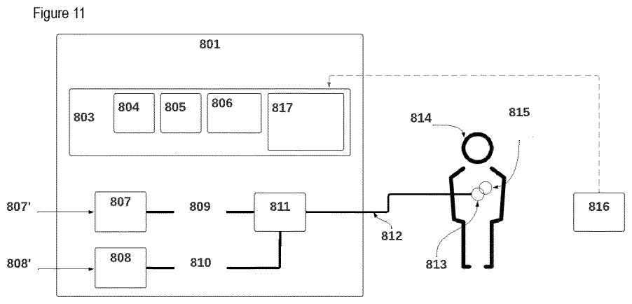

Referring to FIG. 9, in one alternative embodiment, data from coupled sensors 815 is processed by an algorithm within the controller 803 configured to detect suspected infusion reaction and deliver appropriate therapeutic treatment automatically or through intervention by a healthcare provider. In some embodiments, the algorithm utilizes historical data from a single patient to determine whether an infusion reaction is occurring. In some embodiments, the algorithm utilizes historical data from one or more users of the drug delivery system to determine whether an infusion reaction is occurring. In some embodiments, the algorithm utilizes historical data from one or more prior clinical trials with the therapeutic medication being administered to determine whether an infusion reaction is occurring. In some embodiments, the aggregated historical data is analyzed by a machine learning program to improve accuracy or timeliness of infusion reaction identification. In some embodiments, the algorithm uses historical data aggregated from many patients in conjunction with machine learning to compute a probabilistic estimate of whether an infusion reaction is occurring in a present instance. Referring to FIG. 11, in a further embodiment of the embodiment depicted in FIG. 9, data from coupled sensors 815 and patient data 816 is processed by an algorithm 817 within the controller 803 configured to predict, detect, and differentiate suspected infusion reaction and deliver appropriate therapeutic treatment automatically or through intervention by a healthcare provider. In some embodiments, the algorithm utilizes historical data from a single patient to predict the likelihood or severity of an infusion reaction, determine whether an infusion reaction is actively occurring, differentiate one infusion reaction subtype from others, or predict the likelihood of one subtype occurring relative to others. In some embodiments, the algorithm utilizes historical data from one or more users of the drug delivery system to predict the likelihood or severity of an infusion reaction, determine whether an infusion reaction is actively occurring, differentiate one infusion reaction subtype from others, or predict the likelihood of one subtype occurring relative to others. In some embodiments, the algorithm utilizes historical data from one or more prior clinical trials with the therapeutic medication being administered to predict the likelihood or severity of an infusion reaction, determine whether an infusion reaction is actively occurring, differentiate one infusion reaction subtype from others, or predict the likelihood of one subtype occurring relative to others. In some embodiments, the aggregated historical data is analyzed by a machine learning program to improve accuracy or timeliness of infusion reaction prediction, detection, and differentiation. In some embodiments, the algorithm uses historical data aggregated from many patients in conjunction with machine learning to compute a probabilistic estimate of the likelihood or severity of an infusion reaction, whether an infusion reaction is actively occurring, whether one infusion reaction subtype is actively occurring relative to others, or the likelihood of one subtype occurring relative to others.

In one or more embodiments, the drug delivery apparatus or system is configured to halt administration of one or more therapeutic medications immediately if an infusion reaction is detected. In a first alternative embodiment, drug delivery may be halted by the controller 803 interrupting fluidic connection with the tubing set 812. In a second alternative embodiment, drug delivery system may be halted by the controller 803 stopping the fluid pump 811. However, both preceding alternative embodiments are disadvantageous, as no further medications may be administered, including a counteracting emergency medication. In a third alternative and an embodiment, administration of a therapeutic medication may be halted by the controller interrupting fluidic connection between the reservoir 807 and the fluid pump 811, while leaving the fluid pump 811 and tubing set 812 operable to provide administration of a counteracting emergency medication 808 contained in reservoir 808′.

Referring to FIG. 4, in an alternative embodiment, a drug delivery apparatus or system is provided with a reservoir 402 for containing a therapeutic medication, a reservoir 416 containing an emergency medication, and fluidic connections 411, 417 between the reservoirs and a fluid pump 415, a tubing set 405 fluidically connected between the fluid pump 415 and patient interface 406, one or more sensors 407, and one or more sources of patient data 408. Sensor data 410 is communicated to the controller 403 from the sensors 407. In one alternative embodiment, data from sensors 407 is processed by an algorithm within the controller 403 configured to detect suspected infusion reaction and deliver appropriate therapeutic treatment automatically or through intervention by a healthcare provider as described herein. In one alternative embodiment, data from sensors 407 is processed by an algorithm within the controller 403 configured to predict, detect, or differentiate a suspected infusion reaction and deliver appropriate therapeutic treatment automatically or through intervention by a healthcare provider as described herein.

In one or more embodiments, the controller 403 according to one or more embodiments comprises a processor 403a, a memory coupled to the processor 403b, input/output devices 403c coupled to the processor 403a, and support circuits to provide communication between the different components of the system, namely the components of the system described herein. In one or more embodiments, processes to operate the system are stored in the memory 403b as a software routine that, when executed by the processor, causes the system to perform methods described in the present disclosure. In one or more embodiments, the process to operate the system comprises an infusion reaction detection algorithm 403d based on one or more sensor data 410 from one or more patient sensors 407 or patient data 408. In one or more embodiments, the process to operate the system comprises an infusion reaction prediction, detection, and differentiation algorithm 403d based on one or more sensor data 410 from one or more patient sensors 407 or patient data 408. In one or more embodiments, patient data 408 comprises a self-report of symptoms by the patient 407. In one or more embodiments, patient data 408 comprises a self-report of symptoms by the patient 407. In one or more embodiments, patient data 408 is derived from a healthcare provider interaction with a patient 407. In one or more embodiments, the infusion reaction detection algorithm also is configured to respond to a detected infusion reaction in conjunction with the controller 403, whereby one or more emergency medications 416 may be administered, or whereby medication delivery may be halted to a patient 407 as described herein. In one or more embodiments, the processes to operate the system are performed in hardware. In one or more embodiments, the software routine to operate the system may also be stored and/or executed by a second processor that is remotely located from the hardware being controlled by the processor.

In one or more embodiments of the present disclosure, the drug delivery device is configured to halt administration of one or more therapeutic medications of interest immediately if an infusion reaction is detected. In a first alternative embodiment, the drug delivery system 401 may be provided with a fluid flow control 414 configured to interrupt fluidic communication between the fluid pump 415 and the tubing set 405. In a second alternative embodiment, the drug delivery system 401 may be provided with a fluid flow control 412 configured to interrupt the fluid pump 415 and cease all medication delivery to the patient 404.

However, both preceding alternative embodiments have the disadvantage that no further medications may be administered, including a counteracting emergency medication. Thus, in a third alternative and preferred embodiment, the drug delivery system 401 may be provided with a fluid flow control 413 configured to interrupt fluidic communication between the fluid pump 415 and a therapeutic medication reservoir 402, thereby preventing flow of a therapeutic medication 402 causing an infusion reaction, while leaving the fluid pump 415 and tubing set 405 configured to administer a counteracting emergency medication 416 to a patient 404.

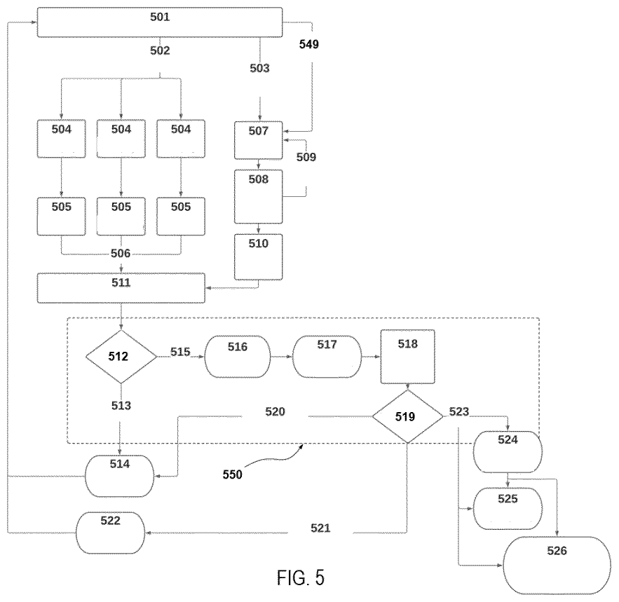

FIG. 5 provides a schematic of an embodiment of the decision-making algorithm within the controller 403 and sensor(s) 407 referenced in FIG. 4, wherein diagnosis and treatment for infusion reactions are supported by the algorithm 403 as a form of decision support for a healthcare provider. The embodiment provides that during medication administration 501, the drug delivery system is configured to detect potential infusion reactions based on one or more of physiologic sensor data 502, in-person or remote observation of the patient's condition 503 by a healthcare provider, and patient self-report 549.

Physiologic data 502 for potential infusion reactions, may include by way of example but not limitation, heart rate, blood pressure, respiratory rate, blood oxygen saturation (SpO2), and temperature, which are collected by way of sensor(s) 407. The plurality of sensors sample the data 504, data is pre-processed 505 using the system's controller and algorithm 403 and the output is aggregated and consolidated 506, also by the controller and algorithm 403.

Sensor data may be supplemented with objective and subjective observation 507 of patients' conditions 503 from physical examination such as flushing, skin reactions, rigors, swelling, urticaria, angioedema, wheezing, stridor, cough, change in voice quality, or loss of consciousness. Sensor data may further be supplemented with data collected from patient interview or self-report 549, including by way of example, headache, shortness of breath, throat closing, diaphoresis, nausea, abdominal or back pain, itching, general anxiety, or self-reported sense of “impending doom.”

Observations of the patient 507 prompt in-person or remote patient interactions and/or patient interviews 508, which are aggregated and evaluated by the healthcare provider in a feedback loop 509 until the patient evaluation is satisfactorily completed, whereupon the healthcare provider uses their clinical judgement and heuristics to arrive at an overall patient assessment 510. Quantitative sensor data 506 and qualitative patient assessment 510 is thus consolidated 511 into an overall patient assessment, which is used to assess whether the patient is experiencing an ongoing infusion reaction 512 and determine the need for emergent treatment.

If an infusion reaction is not suspected 513, administration 501 may be continued at the ongoing administration rate 514. If an infusion reaction is suspected 515, the medication infusion is automatically paused or stopped 516, the patient's situation is immediately escalated, and relevant clinical staff are provided with the appropriate data 517. Upon evaluating the totality of data 517 and the patient 518, the healthcare provider determines whether it is safe to restart the infusion 519. If the healthcare provider determines that the patient is not having an infusion reaction (i.e. “a false alarm”) and it is safe to restart 520, the infusion may be continued at the same administration rate as previously tolerated 514.

If the healthcare provider determines that the patient is having a mild infusion reaction that can be remedied by slowing the infusion rate 521, the infusion may be continued at a reduced rate 522 pre-determined by the healthcare provider by administering medication using the smaller lumen of a multiple-lumen tubing as described elsewhere herein.

If the healthcare provider confirms the patient is experiencing an infusion reaction and determines it is unsafe to restart the infusion 523, they can opt to trigger an optionally provided feature within the drug delivery system to administer one or more emergency medications 524 and optionally call emergency medical services 525. In an alternative embodiment, the emergency medical services 525 are configured to provide a timelier response by virtue of geolocation data 526 provided by the drug delivery apparatus or system.

The treatment algorithm comprising 512, 513, 515, 516, 517, 518, 519, 523, and 524 is provided by way of example and not limitation. More generally, the present disclosure provides one of many alternative evaluation and treatment flows 550, which may be tailored based on the specific therapeutic medication, expected type and severity of infusion reaction, specifics in a prescribed medication order or order set, required counteracting medications, and other clinical considerations.

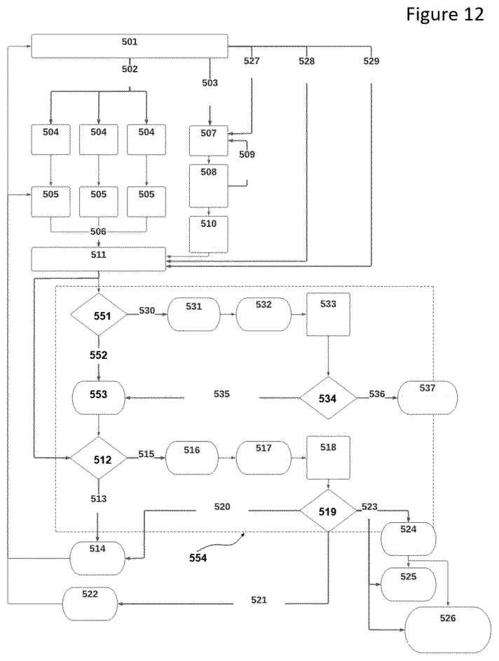

FIG. 12 provides a schematic of an embodiment of the decision-making algorithm within the controller 403, sensor(s) 407, and patient data 408 referenced in FIG. 4, wherein prediction, diagnosis, differentiation, and treatment of infusion reactions are supported by the algorithm 403d as a form of decision support for a patient or healthcare provider. The embodiment provides that before, during, or after medication administration 501, the drug delivery system is configured to predict, detect, or differentiate potential infusion reactions based on one or more of sensor data 502, existing in-person or remote observation of the patient's condition 503 by a healthcare provider, patient self-report 549, patient history, demographics, concomitant medications, and disease characteristics 528, and patient laboratory, telemetric, electrophysiologic, and radiologic measures 529.

Sensor data 502 for potential infusion reactions, may include by way of example but not limitation, heart rate, blood pressure, respiratory rate, blood oxygen saturation (SpO2), temperature, biophysical signals (e.g., electrophysiological, kinematic, thermoregulatory, skin properties, vascular dynamics), biochemical signals (e.g., metabolites, electrolytes, hormones, proteins, other biomarkers present in bodily fluids), and environmental signals (e.g., light, gases, pressure, humidity), which are collected by way of sensor(s) 407. The plurality of sensors sample the data 504, data is pre-processed 505 using the system's controller and algorithm 403 and the output is aggregated and consolidated 506, also by the controller and algorithm 403. In one or more embodiments, sensors 407, sensor data 502, and patient data 408 are selectively chosen based on parameters defined in a drug database or “library” stored within the system's controller 403, thereby enforcing that the most appropriate and relevant data are always collected to predict, detect, or differentiate an infusion reaction to given medication or regimen. In one or more embodiments, the drug database or “library” mentioned above is developed and populated using data collected, generated, and analyzed by the system. In one or more embodiments, sensors 407, sensor data 502, and patient data 408 are de-selected or selectively omitted when they are no longer deemed necessary based on data collected, generated, and analyzed by the system.

Sensor data may be supplemented with objective and subjective observation 507 of patients' conditions 503 from physical examination such as general appearance, flushing, skin reactions, rigors, swelling, urticaria, angioedema, wheezing, stridor, cough, change in voice quality, or loss of consciousness. Sensor data may further be supplemented with data collected from patient interview or self-report 549, including by way of example, headache, shortness of breath, throat closing, diaphoresis, nausea, abdominal or back pain, itching, general anxiety, or self-reported sense of “impending doom.” These data may be further supplemented with data collected about the patient's history, demographics, concomitant medications, and disease characteristics 528, which could include by way of example current or historical information about the patient's age, sex, medical, surgical, social, or family history, symptoms or functional capability, allergies, disease subtype, location, organ involvement, duration, or severity, frequency of disease exacerbations or hospitalizations, current or prior treatments, number and frequency of treatment doses and durations of therapy, and prior history of adverse effects or infusion reactions. These data may be further supplemented with patient laboratory, telemetric, electrophysiologic, and radiologic measures 529, which could include by way of example current or historical complete blood count with differential (e.g., white blood cells with relative proportions of neutrophils, lymphocytes, monocytes, eosinophils, basophils, bands, and blasts, red blood cell number and quality, reticulocytes, hemoglobin, hematocrit, and platelets), chemistry (e.g., sodium, potassium, calcium, magnesium, chloride, bicarbonate, blood urea nitrogen, creatine, glucose), coagulation (e.g., prothrombin time, partial thromboplastin time, international normalized ratio), inflammation (e.g., C-reactive protein, erythrocyte sedimentation rate, plasma viscosity, rheumatoid factor, antinuclear antibody, anti-nuclear factor, anti-double stranded DNA, cyclic citrullinated peptide antibodies, procalcitonin, ferritin, haptoglobin, complement and subtypes, immunoglobulins and subtypes, anti-drug antibodies and subtypes), cytokines (tumor necrosis factors, interleukins, interferons, integrins, clusters of differentiation), lipids (e.g., total cholesterol and subtypes, lipoproteins, triglycerides), allergy studies (e.g., immunoglobulin E, tryptase), urinalysis and urine cytology, microbiology, iron studies, hormone studies, genetic studies, imaging studies (e.g., X-ray, computed tomography, magnetic resonance imaging, positron emission tomography, nuclear scan, bone scan, ultrasound), electrocardiograms, echocardiograms, blood gas studies, pulmonary function tests, biopsies and pathology studies, cancer gene mutation testing, cytogenetic analysis, immunophenotyping, tumor marker tests, tumor bulk, degree and site of cancer metastasis and organ involvement, and cancer staging.

Observations of the patient 507, which may be considered alongside sensor data 502, patient history, demographics, concomitant medications, and disease characteristics 528, and patient laboratory, telemetric, electrophysiologic, and radiologic measures 529, prompt in-person or remote patient interactions and/or patient interviews 508, which are aggregated and evaluated by the healthcare provider in a feedback loop 509 until the patient evaluation is satisfactorily completed, whereupon the healthcare provider uses their clinical judgement and heuristics to arrive at an overall patient assessment 510. Sensor data 506, patient assessment 510, patient history and characteristics 528, and patient measures 529 are thus consolidated 511 into an overall interpretation, which can be used to predict whether the patient is likely to experience an infusion reaction 527, to detect if the patient is experiencing an ongoing infusion reaction 512 once treatment has been initiated, and to determine the need for emergent treatment.

In one or more embodiments, consolidated data 511 is optionally used to predict the likelihood that an infusion reaction will occur 527. If an infusion reaction is not predicted to be likely to occur 552, the infusion is initiated as planned 553 and monitoring for ongoing infusion reaction begins 512. If an infusion reaction is predicted to be likely to occur 530, initiation of the infusion is temporarily precluded 531, and an HCP is notified 532 to assess the patient and available data 533 to determine if the infusion is safe to initiate 534. If a healthcare provider deems that the infusion is safe to initiate (e.g., the benefit of the therapy outweighs the risk of infusion reaction) 535, the infusion will be initiated as planned 553. If a healthcare provider deems that the infusion is not safe to initiate 536, medication administration is prevented until further follow-up 537. In one of more embodiments, prediction of infusion reaction likelihood 527 also provides a probabilistic estimate of the relative likelihood of infusion reaction subtypes (e.g., standard infusion reaction, complement activation-related pseudoallergy, hypersensitivity, anaphylaxis, cytokine release syndrome).

Upon medication administration, if an infusion reaction is not suspected 513, administration 501 may be continued at the ongoing administration rate 514. If an infusion reaction is suspected 515, the medication infusion is automatically paused or stopped 516, the patient's situation is immediately escalated 517, and relevant clinical staff are provided with the appropriate data 518. Upon evaluating the totality of data and the patient 518, the healthcare provider determines whether it is safe to restart the infusion 519. If the healthcare provider determines that the patient is not having an infusion reaction (i.e., “a false alarm”) and it is safe to restart 520, the infusion may be continued at the same administration rate as previously tolerated 514.

If the healthcare provider determines that the patient is having a mild infusion reaction that can be remedied by slowing the infusion rate 521, the infusion may be continued at a reduced rate 522 pre-determined by the healthcare provider by administering medication using the smaller lumen of a multiple-lumen tubing as described elsewhere herein.

If the healthcare provider confirms the patient is experiencing an infusion reaction and determines it is unsafe to restart the infusion 523, they can opt to trigger an optionally provided feature within the drug delivery system to administer one or more emergency medications 524 and optionally call emergency medical services 525. In an alternative embodiment, the emergency medical services 525 are configured to provide a timelier response by virtue of geolocation data 526 provided by the drug delivery apparatus or system.

In one of more embodiments, detection of an infusion reaction 512 and 515 also provides a probabilistic estimate of the relative likelihood of infusion reaction subtypes (e.g., standard infusion reaction, complement activation-related pseudoallergy, hypersensitivity, anaphylaxis, cytokine release syndrome). In other embodiments, detection of an infusion reaction 512 and 515 results in a definitive determination of the specific infusion reaction subtype that is occurring, differentiating it from other subtypes. In either of these embodiments, these data are optionally provided to the healthcare provider 518 evaluating the patient for suspected infusion reaction. In other embodiments, the probabilistic estimate of likely infusion reaction subtypes or definitive determination of the specific subtype that is occurring are used to recommend appropriate responses, including changes in administration rate 522, administration of emergency medications 524, or summoning of emergency medical services 525 and 526. In other embodiments, the probabilistic estimate of likely infusion reaction subtypes or definitive determination of the specific subtype is generated retrospectively (i.e., after the infusion reaction has occurred), such as by way of example in scenarios where more data (e.g., specific laboratory studies) become available post-hoc. This retrospective determination can then be used in future infusion reaction prediction or detection models. In one or more embodiments, the probabilistic estimate is based on Bayesian or conditional probability rather than absolute probability alone.

The algorithm comprising 512, 513, 514, 515, 516, 517, 518, 519, 523, 524, 530, 531, 532, 533, 534, 535, 536, 537, 551, 552 and 553 is provided by way of example and not limitation. More generally, the present disclosure provides one of many alternative evaluation and treatment flows 550, which may be tailored based on the specific therapeutic medication, expected type and severity of infusion reaction, specifics in a prescribed medication order or order set, required counteracting medications, and other clinical considerations.

### Infusion Reaction Response

Contingent administration of emergency medications is particularly provided by the present disclosure, allowing safe administration of medications with propensity to cause side effects or reactions.

FIG. 2E illustrates an exemplary drug delivery apparatus or system configured to contingently administer certain emergency medicines to counteract symptoms and/or to treat systemic infusion reaction caused by administration of one or more therapeutic medications also contained within the system.

In a first alternative embodiment, the drug delivery system is configured to administer one or more emergency medications using the same tubing set lumen used to administer one or more therapeutic medications. A drug delivery apparatus or system 285 is provided with a reservoir 286 for a therapeutic medication 287, a reservoir 288 containing an emergency medication 289, and fluidic connections 290, 291 between the reservoirs and a fluid pump 292, and a single lumen tubing set 293 fluidically connected between the fluid pump 292 and patient interface 295. Medication 287 is administered to the patient. In accordance with the disclosure herein, in the case of a suspected or actual infusion reaction, the emergency medication 221 is administered to the patient 294 through the patient interface 295.

In a second alternative embodiment, the drug delivery system is configured to administer one or more emergency medications in a pre-emptive manner using an alternative lumen than that used to administer one or more therapeutic medications. A drug delivery apparatus or system 285 is provided with a reservoir 286 for a therapeutic medication 287, a reservoir 288 containing an emergency medication 289, and fluidic connections 290, 291 between the reservoirs and a fluid pump 292, and a double lumen tubing set 293′ fluidically connected between the fluid pump 292 and patient interface 295. Medication 287 is administered to the patient using a first medication lumen 297 within the double lumen tubing 293′. In accordance with the disclosure herein, in the case of a suspected or actual infusion reaction, flow of the therapeutic medication 287 is halted within the first medication lumen 297 and the emergency medication 221 is administered through a second medication lumen 298 within double lumen tubing 293′ and into the patient 294 through the patient interface 295.

In some embodiments, the emergency medication is administered in response to a suspected systemic infusion reaction triggered by administration of one or more therapeutic medications. In some embodiments, the emergency medication is administered in response to a patient experiencing an adverse event. In some embodiments, the emergency medication is a reversal agent for one or more therapeutic medications.

In some embodiments, the emergency medication is epinephrine. In some embodiments, the emergency medication is naloxone. In some embodiments, the emergency medication is a corticosteroid. In some embodiments, the emergency medication includes one or more medications selected from the group of hydrocortisone, dexamethasone, or methylprednisolone. In some embodiments, the delivery apparatus or system is configured to reconstitute a lyophilized emergency medication prior to administration. In situations where time may be of the essence, the delivery apparatus or system may be configured to reconstitute a lyophilized emergency medication in an anticipatory fashion, such as when a potential infusion reaction is first detected by a sensor, but before administration has been ordered by a healthcare provider.

In some embodiments, the drug delivery apparatus or system is configured to administer an emergency medication automatically based on predetermined physiologic or clinical criteria. In some embodiments, the drug delivery apparatus or system is configured to administer an emergency medication based on instructions from a remote healthcare provider. In some embodiments, the drug delivery apparatus or system is configured to administer an emergency medication based on instructions from a user proximal to the apparatus or system.

### Clinical Trial Configuration

One primary benefit of embodiments of the drug delivery apparatus or systems disclosed herein is to allow commercial presentations of an approved medication to use the same delivery apparatus or system used in earlier clinical studies, without the need to design, validate, or test a second apparatus or system for commercial presentation. The present disclosure increases flexibility to accommodate a wide variety of pharmacokinetic profiles, even if the behaviors are not known in advance.

Pharmacokinetic (PK) profiles as used herein is a term of convenience, but components of PK profiles are well-understood by those skilled in the art and may include, by way of example but not limitation, bioavailability, Tₘₐₓ, Cₘₐₓ, Area Under Curve (AUC), C_{trough}, absorption rate constant, elimination rate constant, half-life, volume of distribution, clearance, and/or steady state concentrations. As used herein, Cₘₐₓ and C_{trough} are the maximum and minimum concentrations a drug reaches in the systemic circulation after administration of a given dose, respectively. Tₘₐₓ is the time required to reach Cₘₐₓ after administration of a given dose.

FIG. 3A depicts a schematic of the preclinical and clinical development processes for dose determination of a typical parenteral drug with the present disclosure incorporated. In this process, the appropriate tubing set or sets 322 to employ in Phase 1 trials is determined in parallel with and influenced by formulation development 320 and pharmacokinetic modeling 321. Notably, the appropriate tubing set or sets, which govern flow desired rate in the clinical study 324, are decoupled from the dose range, which may be varied independently.

Phase 1 clinical trials are then conducted to establish dose ranges in a manner familiar to those skilled in the art. Tubing set(s) 325 as determined in 322 are supplied to the clinical trial site and are used to conduct the initial Phase 1 trial 324 according to desired clinical trial conditions, including the hypothesized dose ranges 323. Analysis of Phase 1 trial 326 data leads to dosing regimen refinement 327 used to design follow-on clinical trials.

If regimen refinement yields only a single dosing regimen 328, a single appropriate tubing set 330 will be designed for use in Phase 2 studies 330, corresponding to the desired clinical trial condition 339 from pharmacokinetic data and dose evaluation 326. If regimen refinement yields multiple possible dosing regimens 332, one alternative embodiment of the current disclosure provides for an appropriate kit of one or more tubing sets 333 to be designed for use in Phase 2 studies 334, wherein the kit components each correspond to one or more clinical trial conditions 336, 337, or 338.

Once the desired efficacy signal 340 is achieved with one or more dosing regimens, the appropriate tubing set or sets are determined 341 for the Phase 3 clinical trial, and then used in the Phase 3 trial 342 based on prior clinical trial results and corresponding to the desired clinical trial condition. Finally, upon regulatory approval, the appropriate tubing set or sets are selected for the commercial product 344 based on pivotal clinical trial results and the desired commercial presentation.

In an embodiment, during one or more clinical trials, staff select one or more tubing sets from a subset in Phase 1 324 and Phase 2 330 and 334 studies, then select a smaller subset of tubing sets for Phase 3 342 studies. In some embodiments, a smaller subset of tubing sets than those used for Phase 3 342 studies are provided to patients in a commercial presentation of the approved medication. In some embodiments, the same tubing sets used for Phase 3 342 studies are provided in a commercial presentation of the approved medication.

One advantageous aspect of the present disclosure is flexibility to accommodate use in both clinical trials and commercially marketed medications. Special considerations apply to drug delivery apparatus or systems used in clinical trials. Clinical trial data should be accurate, traceable, and reproducible; thus, data integrity is a cornerstone of successful clinical research and is an ethical and regulatory requirement designed to allow confident decision-making regarding approval of medicines.

Clinical trials take place in many different settings, depending on the clinical study phase, specific medication, and patient population. For instance, referring again to FIG. 3A, many Phase 1 studies 304 and Phase 2 studies 309 and 310 are completed at clinical trial sites or in clinic. Phase 3 studies 313 may be completed at clinical trial sites, in clinic, or in the home setting. For Phase 3 studies 313 completed at home, alternative embodiments of the present disclosure are especially advantageous when configured to improve clinical trial data integrity through the incorporation of sensors, controllers, and permanent data storage meeting GCP or other regulatory requirements in a variety of configurations.

Referring to FIG. 8, in an alternative embodiment, the drug delivery system 775 includes one or more sensors 782 coupled to a controller 779 to measure patient 783 vital signs at one or more stages before, during, and after administration of one or more therapeutic medications studied within a clinical trial 776. As medication administration progresses, data from the physiologic sensors 783 are recorded into permanent data storage 785 for later retrieval and analysis 787 by a clinical trial team 784. This provides later analysis of data by the clinical trial team 784 to identify any potential propensity for infusion reactions or other adverse physiologic effect as a result of the medication studied within the clinical trial 776.

In an alternative embodiment, the drug delivery system 775 includes one or more sensors 782 to measure the status of medication administration at one or more stages before, during, and after administration of one or more therapeutic medications studied within a clinical trial 776. As medication administration progresses, sensor 382 data are communicated 781 to a controller 779 and transferred 786 to permanent data storage 785 for later retrieval and analysis 787 by a clinical trial team 784. This provides for later analysis of data by the clinical trial team 784 and verification that each patient received a full medication dose as expected. In an alternative embodiment, the sensors may also be provided on one or more medication reservoirs 776′ containing an investigational therapeutic medication 776.

In an alternative embodiment, the drug delivery system 775 includes one or more sensors 782 to monitor the patient interface throughout administration of one or more investigational therapeutic medications studied within a clinical trial 776. The sensor 782 data are communicated 781 to a controller 779 and transferred 786 to permanent data storage 785 for later retrieval and analysis 787 by a clinical trial team 784. This provides for later analysis of data by the clinical trial team 784 and verification that the medication was, in fact, administered directly to the patient as intended. In some embodiments, the sensor 782 comprises a skin sensor. In some embodiments, the sensor 782 comprises a flow sensor.

In an alternative embodiment, the drug delivery system 775 includes a controller and algorithm 779 to monitor the state of drug delivery system 775 throughout administration of one or more investigational therapeutic medications studied within a clinical trial 776, further communicating 781 any such detected failures to permanent data storage 785 for later retrieval and analysis 787 by a clinical trial team 784. This provides for later analysis of data by the clinical trial team 784 and verification that the drug delivery system 775 operated as intended during administration of an investigational therapeutic medication 776.

### Electronic Health Record Integration

Clinical trials occur in highly controlled settings to minimize confounding variability that could affect data integrity and mask positive or negative pharmaceutical efficacy. Once an investigational therapeutic medication is approved, administration may take place at home, in clinic, or both. In day-to-day patient care, treatment of diseases may be complex, necessitating the coordination of multiple medications, lab tests, and physical visits with a healthcare provider. Health-related information is often stored in an electronic health record (EHR), wherein patient information is centrally stored and accessible to authorized users, such as the patient's doctors, nurses, and pharmacists. By including EHR integration as described herein, the present disclosure provides continuity of care between the clinic and home, which is crucial when medications are given in both settings, as is true in the case of a medication regimen, such as for oncology.

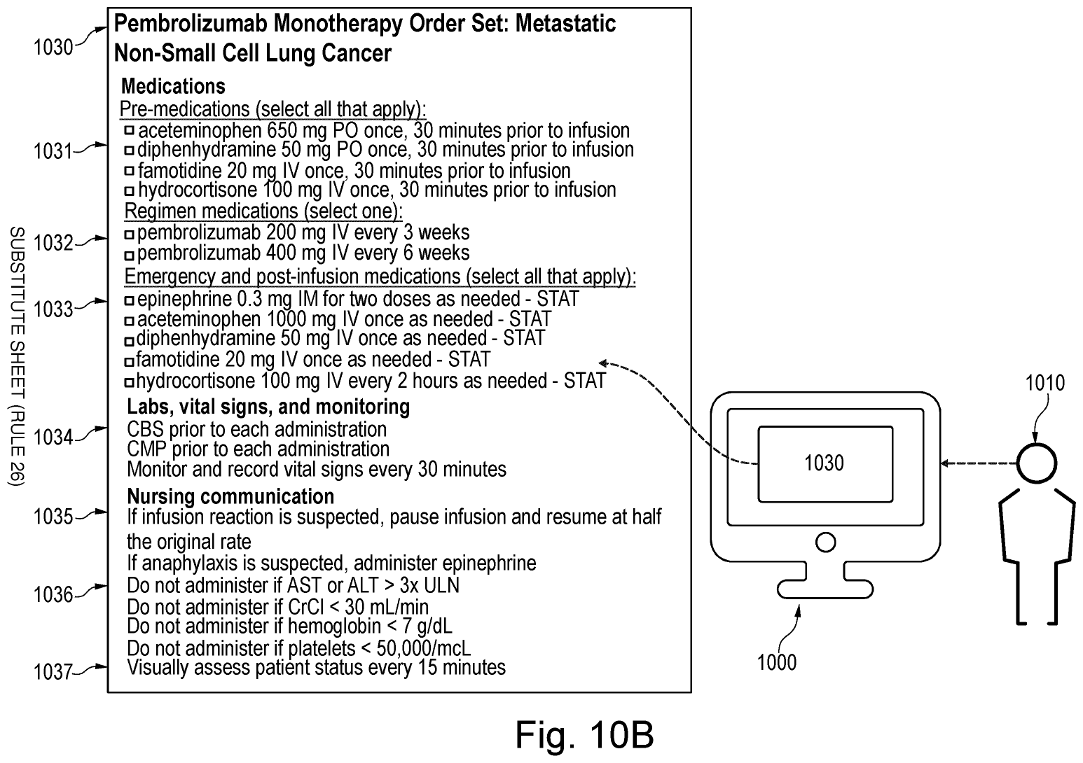

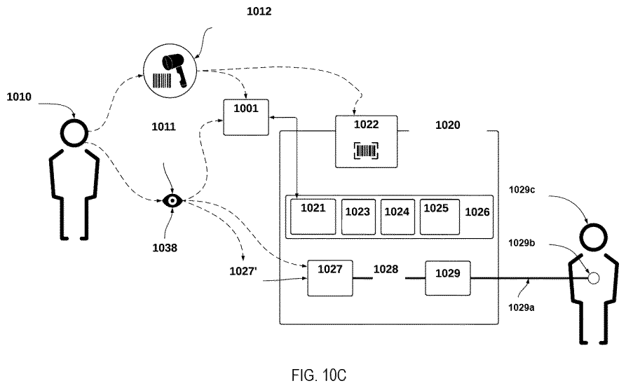

EHRs may also contain orders, which are instructions to care for, diagnose, and treat each patient. Referring to FIG. 10, in one or more embodiments, the drug delivery system 1020 is provided with a controller 1026 that communicates with components of the apparatus via either a wired or wireless connection. In one or more embodiments, the controller according to one or more embodiments comprises a processor 1023, a memory coupled to the processor 1024, input/output devices 1025 coupled to the processor 1023, an EHR interface 1021 coupled to the processor, and support circuits to provide communication between the different components of the system, namely the components of the system described herein. In one or more embodiments, processes to operate the system are stored in the memory 1024 as a software routine that, when executed by the processor, causes the system to perform methods described in the present disclosure. In one or more embodiments, the processes to operate the system are performed in hardware. In one or more embodiments, the software routine to operate the system may also be stored and/or executed by a second processor that is remotely located from the hardware being controlled by the processor.

In some embodiments, the EHR interface 1021 is implemented with a Wi-Fi, wireless local area network (WLAN), Bluetooth, near field communication (NFC), cellular, or internet protocol (IP) connection. In some embodiments, redundant input/output interfaces are provided if one communication interface fails. In some embodiments, the EHR interface 1021 features end to end encryption. In some embodiments, the EHR interface 1021 interface is implemented with an application programming interface (API).

The drug delivery apparatus or system 1020 interfaces with an EHR system 1000 via EHR interface 1021 and is thereby associated with one or more specific medication orders 1001 related to a therapeutic medication 1027. In some embodiments, the association includes corresponding order parameters 1007 and administration time 1008 for a therapeutic medication 1002. In some embodiments, the drug delivery apparatus or system is associated with one or more specific medication orders 1001 contained within EHR system 1000 via EHR interface 1021 and corresponding order parameters contained within the EHR system, wherein the order parameters include an identifying number 1005, prescriber 1009, medication name 1002, and administration parameters 1007 and time 1008. In some embodiments, the drug delivery apparatus or system 1020 is associated with one or more specific medication orders 1030 (shown in FIG. 10B) contained in one or more order sets 1030 contained within the EHR system 1000 via EHR interface 1021.

Order sets may also be provided in EHR systems, comprising aggregation of multiple orders related to a single condition, process, or clinical situation, such as administration of one or more therapies to treat a disease. In some embodiments, the drug delivery system interfaces with an EHR system 1000 via EHR interface 1021 and is thereby associated with one or more specific medication orders 1001 contained in one or more order sets 1030 within an EHR system 1000, wherein the order sets contain administration instructions for one or more therapeutic medications 1032, medications given prior to 1031 and after 1033 one or more therapeutic medications 1032, and/or standing orders related to emergency medication administration 1033. In some embodiments, the drug delivery apparatus or system 1020 is associated with one or more specific medication orders 1001 contained in one or more order sets 1030 within an EHR system 1000, wherein the order sets contain physiologic monitoring instructions 1037 for a given patient.

Prior to administration, orders and order sets are also used in clinical practice to dispense medications to specific patients, and to verify that the proper medicines are dispensed to each patient. In some embodiments, referring to FIG. 10C, the drug delivery apparatus or system 1020 is associated with one or more specific medication orders 1001 within an EHR system 1000, and the drug delivery apparatus or system 1020 contains means by which the contents of reservoir 1027′ holding the therapeutic medication 1027 may be verified at 1011 against the order 1001 by a healthcare provider 1010 prior to dispensing to the patient.

In some embodiments, the drug delivery apparatus or system 1020 is associated with one or more specific medication orders 1001 contained in one or more order sets 1030 within an EHR system 1000, wherein the medication orders are referenced on the drug delivery apparatus or system using a barcode or data matrix 1022 that can be scanned by equipment connected to the EHR system 1000.

In some embodiments, the order set 1030 comprises one or more instructions for administration of one or more therapeutic medications 1032, administration of one or more pre-medications 1031 or post-medications 1032 related, administration of one or more emergency medications 1033, required laboratory values or patient monitoring 1034, or other instructions to nursing 1035, 1036, 1037.

In certain cases, administration of a medication may be subject to specific laboratory values being within specified ranges set forth in one or more medication orders 1001 or order sets 1030. Review of laboratory values may be performed manually by a healthcare provider, or through automated decision support within the EHR system. In some embodiments, the drug delivery apparatus or system 1020 is associated with one or more specific medication orders 1001 contained in one or more order sets 1030 within an EHR system 1000, wherein the order sets permit administration of one or more therapeutic medication(s) 1030 pending review of one or more diagnostic or laboratory criteria 1035 contained elsewhere in the EHR system 1000, wherein the review is completed by a healthcare provider. In some embodiments, the drug delivery apparatus or system 1020 is associated with one or more specific medication orders 1001 contained in one or more order sets 1030 within an EHR system 1000, wherein the order sets permit administration of one or more therapeutic medication(s) 1030 pending review of one or more diagnostic or laboratory criteria 1035 contained elsewhere in the EHR system 1000, wherein the review is completed automatically by a decision support tool also contained within the EHR system 1000.

Medication orders and order sets provide administration instructions, including administration rates. So-called “hard” limits cannot be overridden by a healthcare provider, while so-called “soft” limits may be overridden by a healthcare provider based on professional judgment. Embodiments of the present disclosure allows both types of limits to be implemented. In some embodiments, the drug delivery apparatus or system 1020 is provided with an EHR interface 1021 and is associated with one or more specific medication orders 1001 within an EHR system 1000, wherein the medication orders and EHR interface prohibit administration of one or more therapeutic medication(s) at parameters that are unsafe or clinically inappropriate, and wherein the prohibition may not be overridden by one or more healthcare providers 1010 in the interest of patient safety. In some embodiments, the drug delivery apparatus or system 1020 is provided with an EHR interface 1021 and is associated with one or more specific medication orders 1001 within an EHR system 1000, wherein the medication orders and EHR interface prohibit administration of one or more therapeutic medication(s) 1027 at parameters 1007 that are unsafe or clinically inappropriate, and a means for one or more healthcare providers 1010 to override such prohibition based on clinical judgment.

In some embodiments, the drug delivery apparatus or system 1020 is provided with an EHR interface 1021 and is associated with one or more specific medication orders 1001 within an EHR system 1000, and wherein communication between the EHR interface 1021 and drug delivery apparatus or system 1020 is bi-directional, allowing clinician review 1038 of the order 1001's corresponding parameters and administration progress thereto within the health record system.

In another aspect, the drug delivery system controller herein is provided with an input/output interface to allow communications between the administration location and a remote monitoring service. In some embodiments, all sensor data collected by the drug delivery apparatus or system is communicated to the remote monitoring service by the controller. In some embodiments, a subset of sensor data collected by the drug delivery apparatus or system is communicated to the remote monitoring service by the controller. In some embodiments, the remote monitoring service is manned by a healthcare provider. In some embodiments, the remote monitoring service is a computing apparatus or system. In some embodiments, the remote monitoring service is a healthcare provider aided by a decision support tool implemented in software. In some embodiments, the decision support tool employs a predictive or machine learning algorithm. In some embodiments, the decision support tool is an electronic health record (EHR) system.

In some embodiments, the drug delivery system is programmed based on an order set to monitor specific vital signs contained in one or more orders contained in an order set. In some embodiments, the drug delivery system is programmed to deliver specific therapeutic medications based on an individual medication order or orders contained within an order set. In some embodiments, the drug delivery system is programmed to allow delivery pending availability of certain laboratory test results contained within the EHR. In some embodiments, the drug delivery system is programmed to allow delivery only upon confirmation from the EHR that certain laboratory values are within predefined ranges. In some embodiments, the drug delivery system is programmed to prohibit delivery if certain laboratory values contained within an EHR are unavailable or outside predefined ranges. In some embodiments, the drug delivery system is programmed to prohibit delivery if certain laboratory values contained within an EHR are unavailable or outside predefined ranges unless the prohibition is overridden by a healthcare provider. In some embodiments, the drug delivery system is programmed to prohibit delivery if certain laboratory values contained within an EHR are unavailable or outside predefined ranges unless the prohibition is removed automatically by a decision support tool contained within the EHR.

One or more embodiments of the disclosure utilize at least one controller which can be coupled to various components of the apparatus and systems as described herein. In some embodiments, there are more than one controller connected to the individual components a primary control processor is coupled to each of the separate processors to control the system or apparatus described herein. The controllers may be one of any form of general-purpose computer processor, microcontroller, microprocessor, etc., that can be used in an industrial setting for controlling various delivery and/or treatment regimens.

A controller can have a processor, a memory coupled to the processor, input/output devices coupled to the processor, and support circuits to provide communication between the different electronic components. The memory can include one or more of transitory memory (e.g., random access memory) and non-transitory memory (e.g., storage). The memory, or computer-readable medium, of the processor may be one or more of readily available memory such as random access memory (RAM), read-only memory (ROM), floppy disk, hard disk, or any other form of digital storage, local or remote. The memory can retain an instruction set that is operable by the processor or controller to control parameters and components of the apparatus and methods described herein. The support circuits are coupled to the processor for supporting the processor in a conventional manner. Circuits may include, for example, cache, power supplies, clock circuits, input/output circuitry, subsystems, and the like.

Processes and methods such as treatment regimens may generally be stored in the memory as a software routine that, when executed by the processor, causes the apparatus and systems described herein to perform methods described in the present disclosure. The software routine may also be stored and/or executed by a second processor (not shown) that is remotely located from the hardware being controlled by the processor. Some or all of the method of the present disclosure may also be performed in hardware. As such, the process may be implemented in software and executed using a computer system, in hardware as, e.g., an application specific integrated circuit or other type of hardware implementation, or as a combination of software and hardware. The software routine, when executed by the processor, transforms the general purpose computer into a specific purpose computer (controller) that controls the chamber operation such that the processes are performed.

In some embodiments, the controller has one or more configurations to execute individual processes or sub-processes to perform the methods described herein.

Reference throughout this specification to “one embodiment,” “certain embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments.

Although the disclosure herein has been described with reference to particular embodiments, those skilled in the art will understand that the embodiments described are merely illustrative of the principles and applications of the present disclosure. It will be apparent to those skilled in the art that various modifications and variations can be made to the method and apparatus of the present disclosure without departing from the spirit and scope of the disclosure. Thus, the present disclosure can include modifications and variations that are within the scope of the appended claims and their equivalents.

The drug delivery devices and components described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis, cancer and allergies. Exemplary types of drugs that could be included in the medicament delivery devices described herein include, but are not limited to, antibodies, proteins, fusion proteins, peptibodies, polypeptides, pegylated proteins, protein fragments, protein analogues, protein variants, protein precursors, and/or protein derivatives. Exemplary drugs that could be included in the drug delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-la (multiple sclerosis), sumatriptan (migraines), adalimumab (rheumatoid arthritis), darbepoetin alfa (anaemia), belimumab (lupus), peginterferon beta-la′ (multiple sclerosis), sarilumab (rheumatoid arthritis), semaglutide (type 2 diabetes, obesity), dupilumab (atopic dermatis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the drug delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

In general in this application, unless indicated otherwise, a ‘tubing set’ or ‘tubing’ may comprise one or more tubes, each tube comprising one or more lumens.

### REFERENCE NUMERALS

101 Patient
102 Superior Vena Cava
103 Catheter
104 Patient Interface [Luer Connector(s)]
105 Patient
106 Superior Vena Cava
107 Catheter
108 Patient Interface [Luer Connector(s)]
109 Superior Vena Cava
113 Patient Arm
114 Peripheral vein
115 Catheter
116 Patient Interface [Luer Connector(s)]
120 Catheter
121 Patient
122 Tubing set
123 Patient Skin
124 Port access [Huber] needle
125 Patient Interface (Implanted Port Below Patient Skin)
126A Port Septum
126B Patient interface (port septum of implanted port housing below 127 patient skin 123)
127 Implanted Port Housing
128 Catheter
129 Needle Entry Point (Center of Port Septum)
130 Hand of Patient, Caregiver, or Healthcare Provider
140 SC Needle Assembly
141 Patient Skin
142 SC Needle Cannula
143 SC Needle Point (Hollow)
144 Needle Insertion Grip Affordance
145 Tubing Set
146 Patient Epidermis
147 Patient Dermis
148 Patient Subcutaneous Tissue
149 Patient Muscle Tissue
150 Hand of Patient, Caregiver, or Healthcare Provider
151 IM Needle Assembly
152 Patient Skin
153 Needle Insertion Grip Affordance
154 Tubing Set
155 IM Needle Cannula
156 IM Needle Point (Hollow)
157 Hand of Patient, Caregiver, or Healthcare Provider
158 SC Needle Assembly
159 Patient Skin
160 Angled SC Needle Cannula
170 SC Needle Point (Hollow)
171 Needle Insertion Grip Affordance
172 Tubing Set
173 Patient Epidermis
174 Patient Dermis
175 Patient Subcutaneous Tissue
176 Patient Muscle Tissue
180 Hand of Patient, Caregiver, or Healthcare Provider
181 Two-Part SC Needle Assembly (Soft Cannula and Rigid Inserter Needle)
182 Patient Skin
183 Soft, Flexible SC Administration Cannula
184 Rigid Cannula Inserter Needle
185 Soft, Flexible SC Administration Cannula Tip (Hollow)
186 Needle Insertion Grip Affordance and Inserter Needle Removal Means
187 Tubing Set
188 Hand of Patient, Caregiver, or Healthcare Provider
189 Removed First Part of SC Needle Assembly
190 Removed Rigid Cannula Inserter Needle
191 Retained Second Part of SC Needle Assembly
192 Retained Soft, Flexible SC Administration Cannula
193 Patient Subcutaneous Tissue
194 Soft, Flexible SC Administration Cannula Tip (Hollow)
195 Patient Muscle Tissue
197 Tubing Set
208 Medication Reservoir
209 Medication Reservoir
210 Medication Reservoir
211 Fluidic Connection
212 Fluidic Connection
213 Fluidic Connection
215 Tubing
216 Patient Interface
217 Patient
218 Fluid Pump
219 Outer Housing
220 Therapeutic Medication
221 Therapeutic Medication
222 Therapeutic Medication
224 Medication Reservoir
225 Medication Reservoir
226 Medication Reservoir
227 Pre-Medication(s)
228 Therapeutic Medication
229 Post-Medication(s)
230 Fluidic Connection
231 Fluidic Connection
232 Fluidic Connection
233 Fluid Pump
234 Tubing
235 Patient Interface
236 Patient
240 Outer Housing
241 Medication Reservoir
242 Medication Reservoir
243 Medication Reservoir
244 Medication Reservoir
245 Pre-Administration Flush Solution
246 Therapeutic Medication
247 First Post-Administration Flush Solution
248 Fluidic Connection
249 Fluidic Connection
250 Fluidic Connection
251 Fluidic Connection
252 Fluid Pump
253 Tubing
254 Patient Interface
255 Patient
256 Second Post-Administration Flush Solution
271 Time Delay
272 Time Delay
273 Time Delay
274 Time Delay
275 Time Delay
276 Time Delay
277 Sequential administration
278 Concurrent administration
279 Sequential administration with time-delay start
280 Sequential administration with time delays between one or more medications
281 Concurrent and sequential administration with time delays between one or more medications
282 Beginning of administration
283 End of administration
285 Outer Housing
286 Medication Reservoir
287 Therapeutic Medication of Interest
288 Medication Reservoir
289 Emergency Medication
290 Fluidic Connection
291 Fluidic Connection
292 Fluid Pump
293 Single Lumen Tubing
293′ Double Lumen Tubing
294 Patient Interface
295 Patient
297 First Medication Lumen
298 Second Medication Lumen
318 No; additional Phase 2 study required
319 Desired Efficacy Signal Achieved with One or More Dosing Regimens?
320 Formulation Development (Physical Form Concentration, Volume, Viscosity, Stability, Excipients)
321 Pharmacokinetic Modeling (In Vitro and/or Animal Studies)
322 Determination of tubing sets for Phase 1 clinical trial design
323 Hypothesized Dose Range
324 Initial Human Use Studies with Varying Doses (Phase 1 Clinical Study)
325 Appropriate tubing set(s) used during trial(s) to correspond to desired clinical trial condition(s)
326 Clinical Pharmacokinetic Evaluation and Dose Range Determination
327 Dosing Regimen Refinement for Phase 2 Clinical Study
328 Single Dosing Regimen (flat dose)
329 Determination of tubing set for Phase 2 clinical trial design
330 Early Efficacy Human Use Studies with Single Dose (Phase 2)
331 Single tubing set configuration provided to correspond to desired clinical trial condition
332 Multiple Possible Dosing Regimens (dose banding, weight based dosing)
333 Determination of tubing set(s) for Phase 2 clinical trial design
334 Early Efficiency Human Use Studies with Multiple Doses (Phase 2)
335 Either a single or a plurality of tubing sets provided to correspond to desired clinical trial condition
336 Dose 1
337 Dose 2
338 Dose n
339 Dose 1
340 Yes, positive signal; medication appears effective at one or more doses
341 Determination of tubing set(s) for Phase 3 clinical trial design
342 Pivotal Human Use Studies With Desired Dosing Regimen(s) (Phase 3)
344 Approval and Commercial Use In Various Use Settings Outside of Clinical Trials (at home; in-clinic)
360 Formulation Characteristics (concentration, volume, viscosity, stability, excipients)
361 Pharmacokinetic Modeling
362 Desired Clinical Trial Conditions
363 Nominal calculation using Hagen-Pouiselle or other model
364 Initial design and component selection
365 Mechanical testing of flow rate with initial design and formulated drug product
366 Comparison to desired delivery profile
367 Refined design and component selection
368 Manufacture, Mechanical verification testing and analysis with drug formulation
369 Final set used for clinical trial(s)
380 P=drug pressure (drive force required). L=tubing length. u=viscosity. D=tubing internal diameter, nominal (or toleranced). T=delivery time. V=volume.
391 tubing length
392 tubing internal diameter tolerance
393 tubing nominal internal diameter
401 drug delivery system
402 Therapeutic Medication
403 Controller
403a Processor
403b Memory
403c Input/Output Devices
403d Infusion Reaction Detection/Response Algorithm
404 Patient
405 Tubing
406 Patient Interface
407 Sensor(s)
408 Patient Data
410 Sensor Data
411 Fluidic Connection
412 Fluid Flow Control
413 Fluid Flow Control
414 Fluid Flow Control
415 Fluid Pump
416 Emergency Medication
417 Fluidic Connection
501 Medication Administration
502 (Physiologic) Sensor Data
503 In-Person or Remote Observation of Patient Condition
504 Sensor Sampling
505 Sensor Data Pre-Processing
506 Sensor Data
507 Observations of Patient
508 Patient Interaction, Patient Interview
509 HCP Evaluation, Clarification
510 Heuristic and Clinical Judgment
511 Data Consolidation
512 Suspected infusion reaction?
513 No
514 Continue Administration at prior rate
515 Yes
516 Pause or Stop Administration
517 Notify HCP with Appropriate Data (escalation)
518 HCP Evaluation of Patient & Data
519 Safe to restart the Infusion?
520 Yes (false alarm, no issue)
521 Yes, proceed with caution
522 Continue Administration at reduced rate
523 No
524 Administer one or more emergency medications
525 Summon emergency medical services
526 Summon emergency medical services and direct to specific address via geolocation services
527 Patient Self-Report of Condition
528 Patient History, Demographics, Concomitant Medications, and Disease
Characteristics
529 Patient Laboratory, Telemetric, Electrophysiologic, and Radiologic Measures
530 Yes
531 Do not initiate Infusion
532 Notify HCP with Appropriate Data (escalation)
533 HCP Evaluation of Patent & Data
534 Safe to initiate infusion?
535 Yes (benefit deemed higher than risk)
536 No
537 Prevent Administration and Follow up with HCP
549 Patient Self-Report of Condition
550 Alternative Flow for Different types of infusion reaction
551 Infusion reaction likely?
552 No
553 Initiate Infusion as Planned
554 Alternative Flow for different types of infusion reaction
601 Filter Membrane
602 Undesired Particulate
603 Inflow medication with potential particulate
604 Outflow medication with particulate removed
605 Inflow medication
606 Smoothed inlet to prevent turbulent flow and protein shearing
607 Engineered flow restrictor
608 Outflow at reduced rate
609 Tubing outer diameter (OD)
610 Tubing inner diameter (ID)
640 Tubing
640′ Tubing
641 Medication Lumen
642 Tubing
642′ Tubing Profile
643 Lumen
644 Lumen
645 Lumen
646 Tubing
646′ Tubing Profile
647 Barrier Coating
648 Lumen
775 Outer housing
776 Investigational Therapeutic Medication
776′ Medication reservoir
777 Fluidic Connection
778 Fluid Pump
779 Controller
780 Tubing
781 Communication Means
782 Sensor(s)
783 Patient
784 Clinical Study Team
785 Permanent Data Storage
786 Data Link to Permanent Storage
787 Data Retrieval and Analysis
788 Patient Interface
794 Processor
801 Outer housing
803 Controller
804 Processor
805 Memory
806 Input/Output Devices
807 Therapeutic Medication
807′ Reservoir
808 Therapeutic Medication
808′ Reservoir
809 Fluidic Connection
810 Fluidic Connection
811 Fluid Pump
812 Tubing Set
813 Patient Interface
814 Patient
815 Coupled Sensor(s)
816 Patient Data
817 Infusion Reaction Prediction/Detection/Response Algorithm
1000 Electronic Health Record System (EHR)
1001 (Representative) Medication Order
1002 Therapeutic Medication of Interest
1003 Patient name
1004 Patient identifier
1005 Medication order identifier
1006 Dispensing and verification instructions for Therapeutic Medication of Interest
1007 Administration parameters for Therapeutic Medication of Interest
1008 Administration time and date for Therapeutic Medication of Interest
1009 Medication Prescriber
1010 Healthcare Provider
1011 Healthcare Provider Verification of Reservoir, Medication against Order
1012 Healthcare Provider Verification of Drug Delivery Device against order via barcode
scanning
1013 Laboratory Values
1020 Drug Delivery Device
1021 EMR Interface
1022 Barcode
1023 Processor
1024 Memory
1025 Input/Output Devices
1026 Controller
1027 Therapeutic Medication
1027′ Medication Reservoir
1028 Fluidic Connection
1029 Fluid Pump
1029a Tubing
1029b Patient Interface
1029c Patient
1030 (Representative) Order set associated with Therapeutic Medication of Interest
1031 Medications given prior to administration of therapeutic medication of interest
1032 Therapeutic Medication of Interest
1033 Medicines given post-administration of therapeutic medication of interest, or in emergency
1034 Required labs and patient monitoring instructions
1035 Conditional instructions for nursing
1036 Administration contraindications
1037 Patient assessment instructions
1038 Healthcare Provider Observation of Administration Progress

Some aspects of the present disclosure are outlined in the following clauses:
1. An apparatus configured to deliver a therapeutic medication to a patient, the apparatus comprising:
   a reservoir containing one or more therapeutic medications;
   a patient interface configured to deliver contents of the reservoir into the body of the patient;
   a flexible tubing set in fluid communication with the reservoirs at a proximal end of the flexible tubing set, and the patient interface at a distal end of the flexible tubing set; and
   a fluid pump configured to expel the therapeutic medication from the reservoirs through the flexible tubing set and into the patient interface,
   wherein the flexible tubing set comprises a predetermined length and an internal lumen comprising a consistent internal diameter, the flexible tubing set configured to provide a predetermined, calibrated flow rate based on specific characteristics of the therapeutic medications passing through the internal lumen, the specific characteristics selected from the group consisting of viscosity, shear thinning behaviors, shear thickening behaviors, desired delivery time to the patient, and combinations thereof.
2. The apparatus of embodiment 1, wherein the fluid pump comprises a substantially constant pressure device.
3. The apparatus of embodiment 1, wherein the fluid pump comprises a substantially constant flow device.
4. The apparatus of any of embodiments 1 to 3, wherein the internal diameter of the internal lumen is configured to reduce stresses at an interface of the medication-tubing set and associated aggregation of a protein-based therapeutic medication.
5. The apparatus of any of embodiments 1 to 4, wherein the therapeutic medication is a substantially non-Newtonian fluid.
6. The apparatus of any of embodiments 1 to 5, wherein the therapeutic medication exhibits a non-linear relationship between viscosity and shear stress.
7. The apparatus of any of embodiments 1 to 6, wherein the therapeutic medication exhibits non-linear viscosity changes based on temperature of the medication.
8. The apparatus of any of embodiments 1 to 7, wherein the therapeutic medication is a biologic, recombinant therapeutic protein, gene therapy, monoclonal antibody, antibody-drug conjugate, or fusion protein.
9. The apparatus of any of embodiments 1 to 8, wherein the fluid pump is disposable and designed for one-time use.
10. The apparatus of any of embodiments 1 to 8, wherein the fluid pump is reusable and designed for multiple-time use.
11. The apparatus of any of embodiments 1 to 8, wherein the fluid pump is reusable and designed for use over a course of a single cycle of a medication regimen.
12. The apparatus of any of embodiments 1 to 11, further comprising a controller that is reusable and designed for use over the course of a single cycle of a medication regimen.
13. The apparatus of any of embodiments 1 to 11, further comprising a controller that is disposable and designed for one-time use.
14. The apparatus of any of embodiments 1 to 11, further comprising a controller that is reusable and designed for multiple-time use.
15. The apparatus of any of embodiments 1 to 14, wherein the reservoir is administered by the fluid pump only after elapse of a pre-determined time delay.
16. The apparatus of any of embodiments 1 to 15, wherein the flexible tubing set is configured to provide a flow rate less than a flow rate at which a therapeutic medication may cause an infusion reaction.
17. The apparatus of any of embodiments 1 to 16, further comprising a plurality of flexible tubing sets, and wherein one or more the flexible tubing sets is labeled with an actual flow rate in mL/hour of the therapeutic medication at room temperature based on an experimentally determined concentration-temperature-viscosity relationship.
18. An apparatus configured to deliver one or more therapeutic medications to a patient, the apparatus comprising:
   a plurality of reservoirs, each containing one or more therapeutic medications;
   a patient interface configured to deliver contents of the reservoirs into a body of the patient;
   a flexible tubing set in fluid communication with the reservoirs at a proximal end of the flexible tubing, and the patient interface at a distal end; and
   a fluid pump to expel the therapeutic medication from the reservoirs through the flexible tubing set and into the patient interface,
   wherein the flexible tubing set is provided with a predetermined length and internal lumen comprising a consistent internal diameter configured to provide a predetermined, calibrated flow rate based on specific characteristics of the therapeutic medications passing therethrough, the specific characteristics selected from the group consisting of viscosity, shear thinning behaviors, shear thickening behaviors, desired delivery time to the patient, and combinations thereof.
19. The apparatus of embodiment 18, wherein the fluid pump comprises a substantially constant pressure device.
20. The apparatus of embodiment 18, wherein the fluid pump comprises a substantially constant flow device.
21. The apparatus of any of embodiments 18 to 20, wherein the internal diameter of the internal lumen is configured to reduce stresses at the medication-tubing set interface and associated aggregation of a protein-based therapeutic medication.
22. The apparatus of any of embodiments 18 to 21, wherein the therapeutic medication is a substantially non-Newtonian fluid.
23. The apparatus of any of embodiments 18 to 22, wherein the therapeutic medication exhibits non-linear relationship between viscosity and shear stress.
24. The apparatus of any of embodiments 18 to 23, wherein one of the therapeutic medications exhibit non-linear viscosity changes based on temperature of the medication.
25. The apparatus of any of embodiments 18 to 24, wherein the therapeutic medication is a biologic, recombinant therapeutic protein, gene therapy, monoclonal antibody, antibody-drug conjugate, or fusion protein.
26. The apparatus of any of embodiments 18 to 25, wherein the fluid pump is disposable and designed for one-time use.
27. The apparatus of any of embodiments 18 to 25, wherein the fluid pump is reusable and designed for multiple-time use.
28. The apparatus of any of embodiments 18 to 27, wherein the controller is reusable and designed for use over the course of a single cycle of a medication regimen.
29. The apparatus of any of embodiments 18 to 27, wherein the controller is disposable and designed for one-time use.
30. The apparatus of any of embodiments 18 to 27, wherein the controller is reusable and designed for multiple-time use.
31. The apparatus of any of embodiments 18 to 25 and 28 to 30, wherein the fluid pump is reusable and designed for use over the course of a single cycle of a medication regimen.
32. The apparatus of any of embodiments 18 to 31, wherein the reservoir is administered by the fluid pump only after elapse of a pre-determined time delay.
33. The apparatus of any of embodiments 18 to 32, wherein the flexible tubing set is configured to provide a flow rate less than a flow rate at which one or more therapeutic medications may cause an infusion reaction.
34. The apparatus of any of embodiments 18 to 33, wherein fluid communication between one or more reservoirs and the proximal end of the flexible tubing set is provided by a manifold.
35. The apparatus of any of embodiments 18 to 34, wherein fluid communication between one or more the reservoirs and the proximal end of the flexible tubing set comprises at two or more independent medication lumens, and wherein the diameter of at least a first and second medication lumens are substantially unequal.
36. The apparatus of any of embodiments 18 to 35, wherein fluid communication between one or more the reservoirs and the proximal end of the flexible tubing set comprises at two or more independent medication lumens, and wherein the diameter of at least a first and second medication lumens are substantially equal.
37. The apparatus of any of embodiments 18 to 36, wherein administration of the therapeutic medication from each of the plurality of reservoirs occurs in a predetermined order.
38. The apparatus of any of embodiments 18 to 37, wherein the therapeutic medication from a first of each of the plurality of reservoirs is administered by the fluid pump only after elapse of a pre-determined time delay.
39. The apparatus of any of embodiments 18 to 38, wherein the therapeutic medication from one or more of each of the plurality of reservoirs is administered by the fluid pump only after of a pre-determined time delay that is substantially equal for each of the plurality of reservoirs.
40. The apparatus of any of embodiments 18 to 38, wherein the therapeutic medication from each of the plurality of reservoirs is administered by the fluid pump only after elapse of a pre-determined time delay that is substantially different for each of the plurality of reservoirs.
41. The apparatus of any of embodiments 18 to 40, wherein the therapeutic medications from the plurality of reservoirs are concurrently administered to a patient by the fluid pump.
42. The apparatus of any of embodiments 18 to 40, wherein the therapeutic medications from the plurality of reservoirs are sequentially administered to a patient by the fluid pump, and wherein administration of the therapeutic medication from each of the reservoirs begins only after administration of the therapeutic medication from a preceding reservoir is completed.
43. The apparatus of any of embodiments 18 to 40, wherein the therapeutic medications from the plurality of reservoirs are sequentially administered to a patient by the fluid pump, and wherein administration of the therapeutic medication from a subsequent reservoir begins only after administration of the therapeutic medication from a preceding reservoir begins.
44. The apparatus of any of embodiments 18 to 40, wherein the therapeutic medications from the plurality of reservoirs are sequentially administered to a patient by the fluid pump, and wherein beginning of administration of the therapeutic medication from each of the plurality of reservoirs is separated by one or more time delays.
45. The apparatus of any of embodiments 18 to 44, further comprising a plurality of flexible tubing sets and wherein one or more the flexible tubing sets is labeled with the actual flow rate in mL/hour of the therapeutic medication at room temperature based on an experimentally determined concentration-temperature-viscosity relationship.
46. The apparatus of any of embodiments 18 to 45, further comprising a plurality of flexible tubing sets and wherein one or more the flexible tubing sets is labeled with an ordinal identifier corresponding to one or more of the flow rates of the therapeutic medication at room temperature based on an experimentally determined concentration-temperature-viscosity relationship.
47. The apparatus of any of embodiments 18 to 46, wherein the fluid pump comprises a substantially constant pressure device.
48. The apparatus of any of embodiments 18 to 46, wherein the fluid pump comprises a substantially constant flow device.
49. The apparatus of any of embodiments 18 to 48, wherein the flexible tubing sets provides flow rates less than the rate at which the therapeutic medication may cause an infusion reaction.
50. The apparatus of any of embodiments 18 to 49, further comprising a plurality of flexible tubing sets and wherein the plurality of tubing sets are selected from between two and ten different configurations of predetermined lengths and internal lumen of consistent internal diameters.
51. An apparatus configured to deliver a therapeutic medication to a patient, the comprising:
   one or more reservoirs, each of the one or more reservoirs containing one or more therapeutic medications;
   one or more reservoirs containing either or both of a pre-medication to be administered before or a post-medication to be administered after the one or more therapeutic medications;
   a patient interface configured to deliver contents of the reservoirs into the body of the patient;
   a flexible tubing set in fluid communication with the reservoirs at a proximal end of the flexible tubing set, and a patient interface at a distal end of the flexible tubing set; and
   a fluid pump to expel the therapeutic medication from each of the one or more reservoirs through the flexible tubing set and into the patient interface,
   wherein the flexible tubing set is provided with predetermined length and an internal lumen of consistent internal diameter to provide a specific, calibrated flow rate based on characteristics of the therapeutic medications passing therethrough, the characteristics selected from the group consisting of viscosity, shear thinning behaviors, shear thickening behaviors, desired delivery time to the patient, and combinations thereof.
52. The apparatus of embodiment 51, wherein one or more the pre-medications or the post-medications are selected from the group consisting of an analgesic, an antipyretic, a corticosteroid, an antihistamine, an antiemetic, an antithrombotic, or an antimicrobial.
53. The apparatus of embodiment 51, wherein one or more of the pre-medications or the post-medications comprise a co-formulated antimicrobial and antithrombotic.
54. The apparatus of embodiment 51, wherein one or more the pre-medications or the post-medications are selected from the group consisting of diphenhydramine, acetaminophen, ondansetron, famotidine, hydrocortisone, dexamethasone, and methylprednisolone.
55. The apparatus of embodiment 51, wherein one or more the pre-medications or the post-medications are selected from the group consisting of 0.9% Normal Saline, Heparin Lock Flush solution, 100 U/mL Heparin Lock Flush Solution, or 5000 U/mL Heparin Lock Flush Solution.
56. The apparatus of embodiment 51, wherein one or more the pre-medications or the post-medications is recombinant tissue plasminogen activator (r-TPA).
57. The apparatus of embodiment 51, wherein one or more the post-medications is epinephrine.
58. The apparatus of embodiment 51, wherein one or more the pre-medications is an animal derived, human-derived, or recombinant hyaluronidase enzyme.
59. The apparatus of any of embodiments 51 to 58, wherein fluid communication between one or more the reservoirs and the proximal end of the flexible tubing set comprises two or more independent medication lumens, and wherein a first medication lumen is used to administer a therapeutic medication, and a second medication lumen is used to administer either or both of the pre-medications and post-medications.
60. The apparatus of any of embodiments 51 to 59, wherein administration of the therapeutic medication from each of the one or more reservoirs occurs in a predetermined order.
61. The apparatus of any of embodiments 51 to 60, wherein the therapeutic medication from a first of each of the one or more reservoirs is administered by the fluid pump only after elapse of a pre-determined time delay.
62. The apparatus of any of embodiments 51 to 61, wherein the apparatus is configured to administer one or more pre-medications, followed by administration of one or more therapeutic medications after a pre-determined time delay.
63. The apparatus of any of embodiments 51 to 62, wherein the apparatus is configured to administer one or therapeutic medications, followed by administration of one or more post-medications medications after a pre-determined time delay.
64. The apparatus of any of embodiments 51 to 63, wherein the therapeutic medication from one or more of each of the one or more reservoirs is administered by the fluid pump only after of a pre-determined time delay that is substantially equal for each of the one or more reservoirs.
65. The apparatus of any of embodiments 51 to 63, wherein the therapeutic medication from each of the one or more reservoirs is administered by the fluid pump only after elapse of a pre-determined time delay that is substantially different for each of the one or more reservoirs.
66. The apparatus of any of embodiments 51 to 65, wherein the therapeutic medications from the one or more reservoirs are concurrently administered to a patient by the fluid pump.
67. The apparatus of any of embodiments 51 to 65, wherein the therapeutic medications from the one or more reservoirs are sequentially administered to a patient by the fluid pump, and wherein administration of the therapeutic medication from each of the reservoirs begins only after administration of the therapeutic medication from a preceding reservoir is completed.
68. The apparatus of any of embodiments 51 to 65, wherein the therapeutic medications from the one or more reservoirs are sequentially administered to a patient by the fluid pump, and wherein administration of the therapeutic medication from a subsequent reservoir begins only after administration of the therapeutic medication from a preceding reservoir begins.
69. The apparatus of any of embodiments 51 to 65, wherein the therapeutic medications from the one or more reservoirs are sequentially administered to a patient by the fluid pump, and wherein beginning of administration of the therapeutic medication from each of the one or more reservoirs is separated by one or more time-delays.
70. An apparatus configured to deliver a therapeutic medication to a patient, the apparatus comprising:
   one or more reservoirs, each of the one or more reservoirs containing a therapeutic medication;
   an emergency reservoir containing an emergency medication;
   a patient interface configured to deliver contents of the reservoirs into the body of the patient;
   a flexible tubing set in fluid communication with the reservoirs at a proximal end of the flexible tubing set, and a patient interface at a distal end of the flexible tubing set; and
   a fluid pump to expel the therapeutic medication from each of the one or more reservoirs through the flexible tubing set and into the patient interface, at least one sensor configured in communication with the controller to detect at least one of a physiological aspect of the patient and a physical aspect of the therapeutic medication delivery apparatus;
   a controller having a memory, the controller configured to receive data from the sensor, and to control one or more of start, stop, slow, speed, or continue delivery of the therapeutic medication to the patient in response to data received from the sensor; and
   wherein the flexible tubing set is provided with predetermined length and internal lumen of consistent internal diameter to provide a specific, calibrated flow rate based on characteristics of the therapeutic medications passing therethrough, the characteristics selected from the group consisting of viscosity, shear thinning behaviors, shear thickening behaviors, desired delivery time to the patient, and combinations thereof.
71. The apparatus of embodiment 70, wherein the fluid pump is in communication with a controller, the apparatus thereby configured to halt administration of a therapeutic medication based upon sensor data received by the controller.
72. The apparatus of embodiment 70 or 71, wherein the fluid pump is in communication with a controller, the apparatus thereby configured to halt administration of a therapeutic medication based upon a patient's self-assessment communicated to the controller.
73. The apparatus of any of embodiments 70 to 72, wherein the fluid pump is in communication with a controller, the apparatus thereby configured to begin administration of an emergency medication based upon sensor data received by the controller.
74. The apparatus of any of embodiments 70 to 73, wherein the fluid pump is in communication with a controller, the apparatus thereby configured to begin administration of a therapeutic medication based upon a patient's self-assessment communicated to the controller.
75. The apparatus of any of embodiments 70 to 74, wherein fluid communication between one or more of the reservoirs and the proximal end of the flexible tubing set comprises at least two or more independent medication lumens, a first medication lumen being used to deliver one or more therapeutic medications by the fluid pump, and a second medication lumen being used to administer an emergency medication with the fluid pump if directed by the controller.
76. The apparatus of any of embodiments 70 to 75, wherein the controller is also configured to compare one or more sensor values to a database of sensor values held in the controller memory, the database values representing either of variously safe and unsafe medication administration conditions.
77. The apparatus of embodiment 76, wherein the controller is also configured to halt the fluid pump when an unsafe administration condition is detected by the controller and the one or more sensors.
78. The apparatus of any of embodiments 70 to 77, wherein the controller is also configured to prevent the fluid pump from administering one or more therapeutic medications when an unsafe administration condition is detected by the controller and the one or more sensors.
79. The apparatus of embodiment 78, wherein the controller is also configured to notify a healthcare provider when an unsafe administration condition is detected in a patient using the apparatus by the controller and the one or more sensors.
80. The apparatus of any of embodiments 70 to 79, wherein the controller is also configured to detect onset of an infusion reaction in a patient using the apparatus by the controller and the one or more sensors.
81. The apparatus of embodiment 80, wherein the controller is also configured to notify a healthcare provider when the onset of an infusion reaction is detected in a patient using the apparatus by the controller and the one or more sensors.
82. The apparatus of any of embodiments 70 to 81, wherein the controller is also configured to allow a healthcare provider to remotely start, stop, pause, speed, slow, or continue medication administration when an unsafe administration condition is detected in a patient using the apparatus by the controller and the one or more sensors.
83. The apparatus of any of embodiments 70 to 82, wherein the controller is also configured to allow a healthcare provider to remotely start, stop, pause, speed, slow, or continue medication administration when an infusion reaction is detected in a patient using the apparatus by the controller and the one or more sensors.
84. The apparatus of any of embodiments 70 to 83, wherein the controller is also configured to compare one or more sensor values to one or more sensor values held in the controller memory, the sensor values held in the controller memory representing physiologic data previously associated with imminent or actual infusion reactions to a therapeutic medication.
85. The apparatus of any of embodiments 70 to 84, wherein the controller is also configured to compare one or more sensor values to one or more comparator values held in the controller memory, said comparator values comprising sensor data collected from one or more prior users of the apparatus before, during, or after administration of one or more of the therapeutic medications.
86. The apparatus of embodiment 85, wherein at least one of the comparator values is determined from comprise sensor values from prior administrations of a therapeutic medication to the patient currently receiving one or more medications with the apparatus.
87. The apparatus of embodiment 85 or 86, wherein at least one of the comparator values comprise sensor values from one or more participants in one or more previous human clinical trials conducted with one or more of the therapeutic medications.
88. The apparatus of any of embodiments 70 to 87, wherein the controller is also configured to compare one or more sensor values to one or more sensor values held in the controller memory, the sensor values held in the controller memory representing one or more values contained in an electronic health record.
89. The apparatus of any of embodiments 70 to 88, wherein the controller is also configured to compare one or more sensor values to one or more sensor values held in the controller memory, the sensor values held in the controller memory representing one or more values contained in a medication order for a patient currently receiving one or more medications with the apparatus.
90. The apparatus of any of embodiments 70 to 89, wherein the controller is also configured to compare one or more sensor values to one or more sensor values held in the controller memory, the sensor values held in the controller memory representing one or more values contained in a medication order set for a patient currently receiving one or more medications with the apparatus.
91. The apparatus of any of embodiments 70 to 90, wherein the controller is also configured to prevent the fluid pump from administering one or more therapeutic medications when one or more patient laboratory values are unavailable or outside of safe administration values.
92. The apparatus of any of embodiments 70 to 91, wherein the controller is also configured to prevent the fluid pump from administering one or more therapeutic medications when one or more requisite precursor medications have not been administered to a patient.
93. The apparatus of any of embodiments 70 to 92, further comprising an interface to an electronic health record system.
94. The apparatus of any of embodiments 70 to 93, further comprising an output module interface with an electronic health record, the interface allowing update of a patient's electronic health record with one or more aspects related to delivery of one or more therapeutic medicines.
95. The apparatus of any of embodiments 70 to 94, further comprising an output module interface with an electronic health record, the interface allowing update of a patient's electronic health record with one or more aspects related to delivery of one or more emergency medicines.
96. The apparatus of any of embodiments 93 to 95, wherein the fluid pump is in communication with a controller, the apparatus thereby configured to begin administration of an emergency medication based upon sensor data received by the controller and a contingent medication order for administration of said emergency medication, said order being contained in an electronic health record system.
97. A method for using the apparatus of any of embodiments 70 to 96, comprising:
   collecting sensor data from a patient before administration of a therapeutic medication to that patient using the apparatus;
   identifying, via the sensor data processed by the controller, a current state of the patient prior to medication administration;
   comparing, using the controller and associated computer software, one or more sensor data to one or more predetermined threshold values representing safe medication administration conditions; and
   starting one of administration of a therapeutic medication to a patient if the one or more sensor data are within the one or more predefined threshold values, and preventing administration of a therapeutic medication to a patient if the one or more sensor data are outside the one or more predefined threshold values.
98. A method for using the apparatus of embodiment 97, further comprising notifying a healthcare provider as to the state of the apparatus as determined by the controller, and further comprising the healthcare provider either accepting or overriding a recommendation as to medication administration as determined by the controller.
99. A method for using the apparatus of embodiment 97 or 98, further comprising providing an alert to a user of the apparatus as to the safety of medication administration as determined by the controller.
100. A method for using the apparatus of any of embodiments 97 to 99, further comprising comparing one or more sensor values to predetermined threshold values derived from one or more prior human clinical trials of a therapeutic medication.
101. A method for using the apparatus of any of embodiments 97 to 100, further comprising comparing one or more sensor values to predetermined threshold values derived from one or more prior human clinical trials of a therapeutic medication in conjunction with the apparatus.
102. A method for using the apparatus of any of embodiments 97 to 101, further comprising comparing one or more sensor values to predetermined threshold values derived from one or more prior administrations of a therapeutic medication to a patient presently using the apparatus.
103. A method for using the apparatus of any of embodiments 97 to 102, further comprising comparing one or more sensor values to predetermined threshold values aggregated from prior administrations of a therapeutic medication to one or more patients who have previously received medication with the apparatus.
104. A method for using the apparatus of any of embodiments 70 to 96, comprising:
   administering all or part of a dose of a therapeutic medication to a patient using the apparatus; and
   identifying, via sensor data processed by the controller, a current state of the patient during medication administration; and
   comparing, using the controller and associated computer software, one or more sensor data to one or more predetermined threshold values representing safe medication administration conditions; and
   continuing administration of a therapeutic medication to a patient if the one or more sensor data are within the one or more predefined threshold values, or halting administration of a therapeutic medication to a patient if the one or more sensor data are outside the one or more predefined threshold values.
105. The method of embodiment 104, further comprising slowing an administration rate of a therapeutic medication to a patient.
106. The method of embodiment 104 or 105, further comprising providing an alert to a user of the apparatus as to a status of medication delivery.
107. The method of any of embodiments 104 to 106, further comprising providing an alert to a healthcare provider as to a status of medication delivery using the apparatus.
108. The method of any of embodiments 104 to 107, further comprising updating a patient's electronic health record with a status of medication delivery using the apparatus.
109. A method for using the apparatus of any of embodiments 70 to 96, comprising:
   Administering all or part of a dose of a therapeutic medication to a patient using the apparatus; and
   Identifying, via sensor data processed by the controller, a current state of the patient during medication administration; and
   Comparing, using the controller and associated computer software, one or more sensor data to one or more predetermined threshold values indicative of an infusion reaction to the medication; and
   Continuing administration of a therapeutic medication to a patient if the one or more sensor data do not indicate an infusion reaction is taking place; or
   Halting administration of a therapeutic medication to a patient if the one or more sensor data indicate an infusion reaction is taking place.
110. The method of embodiment 109, further comprising administering an emergency medication when administration of a therapeutic medication is halted.
111. The method of embodiment 109 or 110, further comprising providing an alert to a user of the apparatus as to a status of medication delivery.
112. The method of any of embodiments 109 to 111, further comprising providing an alert to a healthcare provider as to a status of medication delivery using the apparatus.
113. The method of any of embodiments 109 to 112, further comprising updating a patient's electronic health record with a status of medication delivery using the apparatus.
114. An apparatus configured to deliver one or more investigational medicines during a clinical trial at one or more controlled flow rates, the apparatus comprising:
   one or more reservoirs, each of the one or more reservoirs containing an investigational therapeutic medication;
   a patient interface configured to deliver contents of the reservoirs into the body of the patient;
   a flexible tubing set in fluid communication with the reservoirs at a proximal end of the flexible tubing set, and a patient interface at a distal end of the flexible tubing set; and
   a fluid pump to expel the one or more investigational therapeutic medications from each of the one or more reservoirs through the flexible tubing set and into the patient interface,
   at least one sensor in communication with the controller and configured to detect at least one of a physiological aspect of the patient and a physical aspect of the apparatus;
   a controller configured to receive data from the sensor, and to one or more of start, stop, slow, speed, or continue delivery of the therapeutic medication to the patient in response to data received from the sensor; and
   wherein the flexible tubing set is provided with predetermined length and internal lumen of consistent internal diameter to provide a specific, calibrated flow rate based on characteristics of the therapeutic medications passing therethrough, the characteristics selected from the group consisting of viscosity, shear thinning behaviors, shear thickening behaviors, desired delivery time to the patient, and combinations thereof.
115. The apparatus of embodiment 114, further comprising a one or more flexible tubing sets, each corresponding to one or more flow rates, the flow rates corresponding to one or more clinical trial conditions.
116. The apparatus of embodiment 114 or 115, the controller further comprising an interface to a clinical trial data management system.
117. The apparatus of any of embodiments 114 to 116, the controller further configured to update clinical trial data management system with a status of at least one of a physiological aspect of the patient and a physical aspect of the apparatus.
118. The apparatus of embodiment 117, the controller further configured to update clinical trial data management system with a status of at least one of a physiological aspect of the patient and a physical aspect of the apparatus before, during, and after administration of an investigational therapeutic medication.
119. The apparatus of any of embodiments 114 to 118, the controller further configured to receive information from a clinical trial data management system as to the clinical trial condition for a patient using the apparatus.
120. The apparatus of embodiment 119, the controller further configured to verify that the investigational therapeutic medication and tubing set in the apparatus are correct based on the clinical trial condition before beginning administration of the investigational therapeutic medication.
121. The apparatus of embodiment 120, the controller further configured to prevent administration of an investigational therapeutic medication if either of the investigational therapeutic medication or tubing set in the apparatus are incorrect.
122. The apparatus of any of embodiments 114 to 121, wherein the selected flexible tubing set corresponding to an individual patient's clinical trial condition is preassembled to the fluid pump.
123. A method of providing an optimized tubing set for delivery to a patient a therapeutic medication exhibiting substantially non-Newtonian characteristics delivered by a single pump unit at one or more known, preselected, and controlled flow rates, the method comprising:
   identifying one or more desired flow rates of the therapeutic medication for administration to a patient based on desired pharmacokinetics of the therapeutic medication; and
   identifying one or more temperatures at which delivery of the therapeutic medication will occur;
   applying an adjustable constraint to a tubing set with one or more medication lumens situated therein;
   compressing the constraint and tubing interposed therein to a first position;
   instilling the therapeutic medication through an inlet of the tubing set so constrained, at the one or more temperatures at which delivery of the therapeutic medication will occur;
   measuring the flow rate at an outlet of a tubing set so constrained;
   comparing the flow rate at the outlet to the desired flow rate in the tubing;
   compressing the constraint and tubing interposed therein further beyond the first position to a second position if a tested flow rate at the outlet is less than the desired flow rate, or experimentally determining a required fluid pump power to dispense the therapeutic medication if the tested flow rate at the outlet is equal to the desired flow rate; and
   conducting testing to identify a relationship between temperature, viscosity, and concentration of the therapeutic medication in a pharmaceutical formulation for delivery to the patient.
124. The method of embodiment 123, wherein the therapeutic medication is a dilatant or shear-thickening fluid.
125. The method of embodiment 123, wherein the therapeutic medication is a pseudo-plastic or shear-thinning fluid.
126. The method of embodiment 123, wherein the therapeutic medication displays a substantially non-linear concentration-temperature-viscosity relationship.

### ABSTRACT

Apparatus, systems and methods are disclosed, which are configured to deliver a therapeutic medication to a patient. The apparatus, system and methods comprise a reservoir, a patient interface, a tubing set, and a fluid pump, and the components are configured to provide a calibrated flow rate based upon specific characteristics of the therapeutic medications passing through and internal lumen of the tubing set.

1. An apparatus configured to deliver a therapeutic medication to a patient, the apparatus comprising:
a reservoir containing a therapeutic medication;
a patient interface configured to deliver contents of the reservoir into the body of the patient;
a flexible tubing set in fluid communication with the reservoir at a proximal end of the flexible tubing set, and the patient interface at a distal end of the flexible tubing set; and
a fluid pump configured to expel the therapeutic medication from the reservoir through the flexible tubing set and into the patient interface,
wherein the flexible tubing set comprises a predetermined length and an internal lumen comprising a consistent internal diameter, the flexible tubing set configured to provide a predetermined, calibrated flow rate based on specific characteristics of the therapeutic medication passing through the internal lumen, the specific characteristics selected from the group consisting of viscosity, shear thinning behaviors, shear thickening behaviors, desired delivery time to the patient, and combinations thereof.

2. The apparatus of claim 1, wherein the fluid pump comprises a substantially constant pressure device.

3. The apparatus of claim 1, wherein the fluid pump comprises a substantially constant flow device.

4. The apparatus of claim 1, wherein the internal diameter of the internal lumen is configured to reduce stresses at an interface of the medication-tubing set and associated aggregation of a protein-based therapeutic medication.

5. The apparatus of claim 1, wherein the therapeutic medication is a substantially non-Newtonian fluid.

6. The apparatus of claim 1, wherein the therapeutic medication exhibits a non-linear relationship between viscosity and shear stress.

7. The apparatus of claim 1, wherein the therapeutic medication exhibits non-linear viscosity changes based on temperature of the medication.

8. The apparatus of claim 1, wherein the therapeutic medication is a biologic, recombinant therapeutic protein, gene therapy, monoclonal antibody, antibody-drug conjugate, or fusion protein.

9. The apparatus of claim 1, wherein the fluid pump is disposable and designed for one-time use.

10. The apparatus of claim 1, wherein the fluid pump is reusable and designed for multiple-time use.

11. The apparatus of claim 1, wherein the fluid pump is reusable and designed for use over a course of a single cycle of a medication regimen.

12. The apparatus of claim 1, further comprising a controller that is reusable and designed for use over the course of a single cycle of a medication regimen.

13. The apparatus of claim 1, further comprising a controller that is disposable and designed for one-time use.

14. The apparatus of claim 1, further comprising a controller that is reusable and designed for multiple-time use.

15. The apparatus of claim 1, wherein the reservoir is administered by the fluid pump only after elapse of a pre-determined time delay.

16. The apparatus of claim 1, wherein the flexible tubing set is configured to provide a flow rate less than a flow rate at which a therapeutic medication may cause an infusion reaction.

17. The apparatus of claim 1, further comprising a plurality of flexible tubing sets, and wherein one or more the flexible tubing sets is labeled with an actual flow rate in mL/hour of the therapeutic medication at room temperature based on an experimentally determined concentration-temperature-viscosity relationship.

18. The apparatus of claim 1, the apparatus comprising:
a plurality of reservoirs, each containing one or more therapeutic medications.

19. The apparatus of claim 18, wherein the fluid pump is reusable and designed for use over the course of a single cycle of a medication regimen.

20. The apparatus of claim 18, wherein fluid communication between one or more of the plurality of reservoirs and the proximal end of the flexible tubing set is provided by a manifold.
